(19) United States
(12) Patent Application Publication
(10) Pub. No.: US 2022/0064214 A1
(43) Pub. Date: Mar. 3, 2022

(54) NOVEL PEPTIDES AND USES THEREOF

(71) Applicant: CENNA BIOSCIENCES INC., La Jolla, CA (US)

(72) Inventor: Nazneen DEWJI, San Diego, CA (US)

(73) Assignee: CENNA BIOSCIENCES INC., La Jolla, CA (US)

(21) Appl. No.: 17/420,927

(22) PCT Filed: Jan. 6, 2020
(86) PCT No.: PCT/US2020/012323

§ 371 Date: Jul. 6, 2021

Related U.S. Application Data
(60) Provisional application No. 62/789,114, filed on Jan. 7, 2019.

(51) Int. Cl. C07K 7/06; A61P 25/28

(52) U.S. Cl. CPC C07K 7/06; A61K 38/00; A61P 25/28

(57) ABSTRACT

A peptide comprising a sequence of Ac-DEEEDEEL-NH₂, a sequence of dEEEDEEL-NH₂ or a sequence of Ac-dEEEDEEL-NH₂, a pharmaceutical composition comprising the peptide, and use of the peptide for treating a disease or disorder.

### CROSS-REFERENCE TO RELATED APPLICATIONS

This application claims the benefit of U.S. Provisional Patent Application No. 62/789,114 filed Jan. 7, 2019, which is incorporated by reference herein in its entirety.

This invention was made with government support under R44AG043278 awarded by National Institutes of Health (NIH). The government has certain rights in the invention.

### SEQUENCE LISTING

This application incorporates by reference a Sequence Listing submitted with this application as a text format, entitled 14461-003-228_SEQ_LISTING.txt, created on Jan. 3, 2020 having a size of 4,096 bytes.

### 1. FIELD

Provided herein are novel peptides that bind to preselinin 1 and/or preselinin 2 binding-domains on β-APP and pharmaceutical compositions comprising the same, methods, and uses thereof.

### 2. BACKGROUND

Neurodegenerative disorders are progressive diseases characterized by loss of specific neuronal populations and loss of the corresponding neuronal functions. Among the varieties of neurodegenerative discorders, Alzheimer's disease (AD) is the most prevalent, characterized by progressive memory impairment and cognitive decline. The most defining neuropathological hallmarks of AD are the presence of amyloid plaques and neurofibrillary tangles in affected brains. The former is formed by extracellular deposits of amyloid β (Aβ), which is a proteolytic product of Amyloid Precusor Protein (APP; see Serrano-Pozo et al., 2011, Cold Spring Harb Perspect Med. 1,1, a006189) after cleavage of γ-secretase (see Zhang et al., 2011, Mol. Brain 4, 3).

Presenilin 1 and 2 (PS-1 and PS-2) are catalytic components of γ-secretase. More than 150 different mutations in PS-1 and PS-2 have been identified in AD epidemiology studies that were associated with modulating the generation of Aβ peptides (see Haas et al., 2012, Cold Spring Harb Perspect Med. 2(5): a006270). The production and accumulation of Aβ triggers a cascade of neurodegenerative events resulting in the formation of neuritic plaques and intra-neuronal fibrillary tangles and neuronal loss in AD (see Greenfield et al., 2000, Front. Biosci. 5, D72-D83; Golde, 2005, Brain Pathol. 15, 84-87).

### 3. SUMMARY OF THE DISCLOSURE

In one aspect, provided herein is a peptide comprising (i) a sequence of Ac-DEEEDEEL-NH₂ (SEQ ID NO:3), wherein the N-terminal amino acid Asp is acetylated and the C-terminal amino acid Leu is amidated; (ii) a sequence of dEEEDEEL-NH₂ (SEQ ID NO:4), wherein the N-terminal amino acid Asp is a D-amino acid and the C-terminal amino acid Leu is amidated; or (iii) a sequence of Ac-dEEEDEEL-NH₂ (SEQ ID NO:5), wherein the N-terminal amino acid Asp is a D-amino acid and is acetylated and the C-terminal amino acid Leu is amidated.

In some embodiments, the peptide comprises a sequence of Ac-DEEEDEEL-NH₂ (SEQ ID NO:3). In some embodiments, the peptide comprises a sequence of dEEEDEEL-NH₂ (SEQ ID NO:4). In yet other embodiments, the peptide comprises a sequence of Ac-dEEEDEEL-NH₂ (SEQ ID NO:5).

In another aspect, provided herein is a peptide comprising a first domain comprising a sequence of SEQ ID NO: 3, SEQ ID NO:4 or SEQ ID NO:5, and a second domain.

In some embodiments, the second domain comprises an Fc domain. In other embodiments, the second domain comprises a purification peptide.

In another aspect, provided herein is a pharmaceutical composition comprising the peptide provided herein and a pharmaceutically acceptable excipient.

In another aspect, provided herein is a method of attenuating the binding of beta-Amyloid precursor protein (β-APP) with presenilin-1 (PS-1) and/or presenilin-2 (PS-2) in a cell comprising contacting a cell with the peptide or the pharmaceutical composition provided herein.

In another aspect, provided herein is a method of attenuating the production of amyloid β in a cell comprising contacting a cell with the peptide or the pharmaceutical composition provided herein.

In another aspect, provided herein is method of attenuating the amyloid β level in a subject comprising administering to the subject a therapeutically effective amount of the peptide or the pharmaceutical composition provided herein.

In some embodiments, the amyloid β is amyloid β 40. In other embodiments, the amyloid β is amyloid β 42.

In yet another aspect, provided herein is a method of treating a disease or disorder in a subject, comprising administering to the subject a therapeutically effective amount of the peptide or the pharmaceutical composition provided herein.

In some embodiments, the disease or disorder is an amyloid (or amyloid (3) related disease or disorder, a disease or disorder associated with amyloid fibril formation, aggregation or deposition, a neurological disease, or a neurodegenerative disease.

In some embodiments, the disease or disorder is selected from a group consisting of Alzheimer's disease, Parkinson's disease, traumatic brain injury, amyotrophic lateral sclerosis, multiple sclerosis, and dementia.

In some embodiments, the disease or disorder is dementia or a dementia related disease or disorder selected from a group consisting of frontotemporal dementia, fronto-temporal degeneration associated with Pick's disease, vascular dementia, corticobasal degeneration, ischemic vascular dementia (IVD), Lewy body dementia, and Alzheimer's dementia.

In some embodiments, the disease or disorder is an ocular disorder or Downs syndrome. In some embodiments, the ocular disorder is related to Alzheimer's disease. In other embodiments, the ocular disorder is macular degeneration. In some embodiments, the macular degeneration is age-related macular degeneration (AMD).

In some embodiments, the disease or disorder is selected from a group consisting of transmissible spongiform encephalopathies, cerebral amyloid angiopathy, hereditary cerebral hemorrhage with amyloidosis, mild cognitive impairment, sporadic inclusion body myositis and age-related macular degeneration.

In yet another aspect, provided herein is a method for improving memory in a subject, comprising administering to a subject a therapeutically effective amount of the peptide or the pharmaceutical composition provided herein.

Provided herein is a method of attenuating an amyloid (3-induced activity in a subject, comprising administering to the subject a therapeutically effective amount of the peptide or the pharmaceutical composition provided herein.

Provided herein is a method of inhibiting an amyloid (3-induced activity in a subject, comprising administering to the subject a therapeutically effective amount of the peptide or the pharmaceutical composition provided herein.

Provided herein is a method of attenuating an amyloid (3-induced activity in a cell, comprising contacting the cell with a therapeutically effective amount of the peptide or the pharmaceutical composition provided herein.

Provided herein is a method of inhibiting the production of a tau protein in a subject, comprising administering to the subject a therapeutically effective amount of the peptide or the pharmaceutical composition provided herein. In one embodiment, the tau protein is a phosphorylated tau protein. In another embodiment, the tau protein is a hyperphosphorylated tau protein.

Provided herein is a method of attenuating the tau protein level in a subject, comprising administering to the subject a therapeutically effective amount of the peptide or the pharmaceutical composition provided herein. In one embodiment, the tau protein is a phosphorylated tau protein. In another embodiment, the tau protein is a hyperphosphorylated tau protein.

Provided herein is a method of inhibiting the production of a tau protein in a cell, comprising contacting the cell with an effective amount of the peptide or the pharmaceutical composition provided herein. In one embodiment, the tau protein is a phosphorylated tau protein. In another embodiment, the tau protein is a hyperphosphorylated tau protein.

Provided herein is a method of attenuating a tau protein-induced activity in a subject, comprising administering to the subject a therapeutically effective of the peptide or the pharmaceutical composition provided herein.

In some embodiments, the subject is a human subject.

### 4. BRIEF DESCRIPTION OF THE DRAWINGS

FIGS. 1A-1D show “mean concentration versus time” profile of P8 (circle) and 8M2D (square) in rat plasma and CSF following IV (FIG. 1A and FIG. B) and SC (FIG. 1C and FIG. 1D) administration.

FIG. 2 shows mean 8M2D (mP8) plasma concentrations following SC administration in transgenic mice.

FIG. 3 depicts the results of the pharmacodynamics (PD) assay described in Example 4 demonstrating that 8M2D can reduce Aβ 40 more effectively than P8 and 8M1D, in which rats were subcutaneously adminstered with different peptides, and Aβ 40 in plasma was quantified at different time points post dosing.

FIG. 4A showns the concentrations of P8 in various brain regions. FIG. 4B shows Aβ analysis in CSF of APP transgenic mice following P8 (left panel) and 8M2D (middle and right panels) administration.

### 5. DETAILED DESCRIPTION

The present disclosure provides novel peptides that bind specifically to the preselinin 1 and/or 2 binding-domains on β-APP, pharmaceutical compositions comprising same, methods, and uses thereof. More specifically, the present disclosure provides peptides that may inhibit the processing of β-APP into AB, pharmaceutical compositions comprising the same, methods and uses thereof.

Neurodegenerative disorders are progressive diseases characterized by loss of specific neuronal populations and loss of the corresponding neuronal functions. The most consistent risk factor for developing a neurodegenerative disorder is aging (see Tanner, 1992, Neurol. Clin. 10:317-329). These neurodegenerative disorders include, for example, Alzheimer disease (AD), Parkinson disease (PD), Huntington disease (HD), and amyotrophic lateral sclerosis (ALS) (see Przedborski et al., 2003, J Clin Invest. 111(1): 3-10).

Alzheimer's disease (AD) is the most prevalent neurodegenerative disorder in aging populations, characterized by progressive memory impairment and cognitive decline. The most defining neuropathological hallmarks of AD are the presence of amyloid plaques and neurofibrillary tangles in affected brains. The former is formed by extracellular deposits of amyloid β (Aβ), which is a proteolytic product of Amyloid Precusor Protein (APP; see Serrano-Pozo et al., 2011, Cold Spring Harb Perspect Med. 1,1, a006189). A subset (<10%) of AD, familial early-onset AD (FAD), is an autosomal dominant disorder with clinically identifiable AD symptoms at younger ages than sporadic cases. Mutations in the genes encoding presenilins (PS-1 and PS-2) on chromosome 1 and 14 were found to be causative in the majority of FAD cases (see Levy-Lahad et al., 1995, Science 269, 973-977; Sherrington et al., 1995, Nature 375, 754-760). Because the differences in pathological features between FAD and sporadic AD cases are minimal, it is believed that PS-1 and PS-2 are involved in the general pathogenesis of AD, including the sporadic cases that constitute the majority of AD (see Zhang et al., 2014, Front Cell Neurosci. 8, 427).

It is discovered that presenilin 1 and 2 are catalytic components of γ-Secretase. γ-secretase belongs to a family of intramembrane cleaving proteases (i-CLiPs), whose members enzymatically cleave their substrates within the plane of the lipid bilayer in a process termed regulated intramembrane proteolysis (Brown et al., 2000, Cell 100, 391-398; Kopan and Ilagan, 2004, Nat. Rev. Mol. Cell Biol. 5, 499-504). γ-secretase has been proven to cleave multiple subtrates, such as APP. Specifically, APP is cleaved firstly by β-secretase into soluble APPβ (sAPPβ) and the carboxyl terminal fragment (CTF) of APP. The CTF is further cleaved by γ-secretase to generate Aβ and the intracellular domain of APP (AICD). γ-secretase-mediated cleavage takes place at various sites, resulting in different species of Aβ, such as Aβ 40 and Aβ 42 (see Zhang et al., 2011, Mol. Brain 4, 3).

Defects in PS-1 or PS-2 affect the activity of γ-secretase, thus vary the generation of Aβ peptides and change the relative amount of Aβ42 versus Aβ40 (see Haas et al., 2012, Cold Spring Harb Perspect Med. 2(5): a006270). The production and accumulation of Aβ triggers a cascade of neurodegenerative events resulting in the formation of neuritic plaques and intra-neuronal fibrillary tangles and neuronal loss in various diseases or disorders such as AD (see Greenfield et al., 2000, Front. Biosci. 5, D72-D83; Golde, 2005, Brain Pathol. 15, 84-87).

In addition to APP, γ-secretase also cleaves many other type I transmembrane protein substrates: Notch (see De Strooper et al., 1999, Nature 398, 518-522), E-cadherin (see Marambaud et al., 2002, EMBO J. 21, 1948-1956), ErbB4 (see Ni et al., 2001, Science 294, 2179-2181), CD44 (see Lammich et al., 2002, J. Biol. Chem. 277, 44754-44759), tyrosinase (see Wang et al., 2006, Proc. Natl. Acad. Sci. USA 103, 353-358), TREM2 (see Wunderlich et al., 2013, J. Biol. Chem. 288, 33027-33036) and Alcadein (see Hata et al., 2012, Mol. Neurodegener. 7:16. 10.1186/1750-1326-7-16) among others, suggesting the participation of γ-secretase in a vast range of biological activities (see Zhang et al., 2014, Front Cell Neurosci. 8, 427). Therefore, it is not surprising that drugs that were designed to target γ-secretase did not show much promise in clinical trials. For example, in a phase III clinical trial of a γ-secretase inhibitor (GSI) semagacestat, it had no effects on improving cognitive functions, with patients receiving the higher dose had significant worsening of functional ability. It was also associated with more adverse events including skin cancers and infections (see Doody et al., 2013, N. Engl. J. Med. 369, 341-350). Even for a novel class of γ-secretase inhibitors that selectly bind to APP while sparing Notch, such as, BMS-708,163 from Bristol-Myers-Squibb (BMS) (see Gillman et al., 2010, ACS Med Chem Lett 1: 120-124), and PF-3,084,014 from Pfizer (see Lanz et al., 2010, J Pharmacol Exp Ther 334: 269-277; De Strooper et al., 2012, Cold Spring Harb Perspect Med. 2(1): a006304), no progress in terms of moving further in clinical trials was seen. Later, the industry shifted to develop γ-secretase modulators (GSMs) after the failures of GSIs. The first generation of GSMs showed low potency and undesired neuropharmacokinetic properties (see Zhang et al., 2014, Front Cell Neurosci. 8, 427). E2012, the only second generation of GSM that entered clinical development, had no updates since 2008 from its sponsor Eisai Medical Research Inc. Thus, there remains a need for new therapeutic agents capable of specifically interfering the interaction of γ-secretase and PAPP without affecting other substrates.

As demonstrated in Section 6 below, in certain embodiments, the peptides provided herein prevent the binding of γ-secretase to βAPP through competitively occupying the enzyme binding sites on βAPP. Since the peptides provided herein bind to the substrate βAPP instead of targeting the enzyme γ-secretase, it confers the peptide incomparable specificity that will very likely translate into superior efficacy and safety profiles. The peptides provided herein reduce Aβ40 effectively (e.g., 24 h after a one-time subcutaneous administration, 8M2D showed a long-lasting effect on reducing Aβ 40 by about 27%). The peptides provided herein are also relatively stable in human fresh plasma (e.g., the retention rate was about 50% to 90% after an incubation of 24 hr). Moreover, the peptides provided herein have robust pharmacokinetic exposures when administered in rats through different routes. The above mentioned and other properties make the peptides provided herein advantageous candidates for treating various diseases or conditions that are related to Aβ, e.g., AD.

### 5.1 Definitions

To facilitate understanding of the disclosure set forth herein, a number of terms are defined below.

Techniques and procedures described or referenced herein include those that are generally well understood and/or commonly employed using conventional methodology by those skilled in the art.

Unless otherwise defined herein, technical and scientific terms used in the present description have the meanings that are commonly understood by those of ordinary skill in the art. For purposes of interpreting this specification, the following description of terms will apply and whenever appropriate, terms used in the singular will also include the plural and vice versa. In the event that any description of a term set forth conflicts with any document incorporated herein by reference, the description of the term set forth below shall control.

The terms “polypeptide” and “peptide” and “protein” are used interchangeably herein and refer to polymers of amino acids of any length. The polymer may be linear or branched, it may comprise modified amino acids, and it may be interrupted by non-amino acids. The terms also encompass an amino acid polymer that has been modified naturally or by intervention; for example, disulfide bond formation, glycosylation, lipidation, acetylation, phosphorylation, or any other manipulation or modification. Also included within the definition are, for example, polypeptides containing one or more analogues of an amino acid, including but not limited to, unnatural amino acids, as well as other modifications known in the art.

The terms “binds” or “binding” refer to an interaction between molecules including, for example, to form a complex. Interactions can be, for example, non-covalent interactions including hydrogen bonds, ionic bonds, hydrophobic interactions, and/or van der Waals interactions. A complex can also include the binding of two or more molecules held together by covalent or non-covalent bonds, interactions, or forces. The strength of the total non-covalent interactions between two peptides is the affinity of one peptide to the other peptide. The ratio of dissociation rate (koff) to association rate (kon) of a peptide to another peptide (koff/kon) is the dissociation constant KD, which is inversely related to affinity. The lower the KD value, the higher the affinity. The dissociation constant KD for a peptide to its target provided herein can be determined using any method provided herein or any other method well known to those skilled in the art.

The term “variant” when used in relation to a peptide may refer to a peptide or polypeptide comprising one or more (such as about 1 to about 5) amino acid sequence substitutions, deletions, and/or additions as compared to a native or unmodified sequence. As used herein, the term “variant” also includes a “modification” of an amino acid residue/position in a peptide, and a “modification” of an amino acid residue/position refers to a change of a primary amino acid sequence as compared to a starting amino acid sequence, wherein the change results from a sequence alteration involving said amino acid residue/position. For example, typical modifications include substitution of the residue with another amino acid (e.g., a conservative or non-conservative substitution) or a isomeric form thereof, chemical modification of the amino acid residue, insertion of one or more (e.g., generally fewer than 5, 4, or 3) amino acids adjacent to said residue/position, and/or deletion of said residue/position.

The term “identity” or “homology” refers to a relationship between the sequences of two or more polypeptide molecules or two or more nucleic acid molecules, as determined by aligning and comparing the sequences. “Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN, or MEGALIGN (DNAStar, Inc.) software. Those skilled in the art can determine appropriate parameters for aligning sequences, including any algorithms needed to achieve maximal alignment over the full length of the sequences being compared.

The term “Fc region” herein is used to define a C-terminal region of an immunoglobulin heavy chain, including, for example, native sequence Fc regions, recombinant Fc regions, and variant Fc regions. Although the boundaries of the Fc region of an immunoglobulin heavy chain might vary, the human IgG heavy chain Fc region is often defined to stretch from an amino acid residue at position Cys226, or from Pro230, to the carboxyl-terminus thereof. The C-terminal lysine (residue 447 according to the EU numbering system) of the Fc region may be removed, for example, during production or purification of the antibody, or by recombinantly engineering the nucleic acid encoding a heavy chain of the antibody. Accordingly, a composition of intact antibodies may comprise antibody populations with all K447 residues removed, antibody populations with no K447 residues removed, and antibody populations having a mixture of antibodies with and without the K447 residue. A “functional Fc region” possesses an “effector function” of a native sequence Fc region. Exemplary “effector functions” include C1q binding; CDC; Fc receptor binding; ADCC; phagocytosis; downregulation of cell surface receptors (e.g., B cell receptor), etc. Such effector functions generally require the Fc region to be combined with a binding region or binding domain (e.g., an antibody variable region or domain) and can be assessed using various assays known to those skilled in the art. A “variant Fc region” comprises an amino acid sequence which differs from that of a native sequence Fc region by virtue of at least one amino acid modification (e.g., substituting, addition, or deletion). In certain embodiments, the variant Fc region has at least one amino acid substitution compared to a native sequence Fc region or to the Fc region of a parent polypeptide, for example, from about one to about ten amino acid substitutions, or from about one to about five amino acid substitutions in a native sequence Fc region or in the Fc region of a parent polypeptide. The variant Fc region herein can possess at least about 80% homology with a native sequence Fc region and/or with an Fc region of a parent polypeptide, or at least about 90% homology therewith, for example, at least about 95% homology therewith.

The term “vector” refers to a substance that is used to carry or include a nucleic acid sequence, including for example, a nucleic acid sequence encoding a peptide provided herein or the parental peptide thereof, in order to introduce a nucleic acid sequence into a host cell. Vectors applicable for use include, for example, expression vectors, plasmids, phage vectors, viral vectors, episomes, and artificial chromosomes, which can include selection sequences or markers operable for stable integration into a host cell's chromosome. Additionally, the vectors can include one or more selectable marker genes and appropriate expression control sequences. Selectable marker genes that can be included, for example, provide resistance to antibiotics or toxins, complement auxotrophic deficiencies, or supply critical nutrients not in the culture media. Expression control sequences can include constitutive and inducible promoters, transcription enhancers, transcription terminators, and the like, which are well known in the art. When two or more nucleic acid molecules are to be co-expressed, both nucleic acid molecules can be inserted, for example, into a single expression vector or in separate expression vectors. For single vector expression, the encoding nucleic acids can be operationally linked to one common expression control sequence or linked to different expression control sequences, such as one inducible promoter and one constitutive promoter. The introduction of nucleic acid molecules into a host cell can be confirmed using methods well known in the art. Such methods include, for example, nucleic acid analysis such as Northern blots or polymerase chain reaction (PCR) amplification of mRNA, immunoblotting for expression of gene products, or other suitable analytical methods to test the expression of an introduced nucleic acid sequence or its corresponding gene product. It is understood by those skilled in the art that the nucleic acid molecules are expressed in a sufficient amount to produce a desired product and it is further understood that expression levels can be optimized to obtain sufficient expression using methods well known in the art.

The term “host” as used herein refers to an animal, such as a mammal (e.g., a human).

The term “host cell” as used herein refers to a particular subject cell that may be transfected with a nucleic acid molecule and the progeny or potential progeny of such a cell. Progeny of such a cell may not be identical to the parent cell transfected with the nucleic acid molecule due to mutations or environmental influences that may occur in succeeding generations or integration of the nucleic acid molecule into the host cell genome.

An “isolated nucleic acid” is a nucleic acid, for example, an RNA, DNA, or a mixed nucleic acid, which is substantially separated from other genome DNA sequences as well as proteins or complexes such as ribosomes and polymerases, which naturally accompany a native sequence. An “isolated” nucleic acid molecule is one which is separated from other nucleic acid molecules which are present in the natural source of the nucleic acid molecule. Moreover, an “isolated” nucleic acid molecule, such as a cDNA molecule, can be substantially free of other cellular materials, or culture medium when produced by recombinant techniques, or substantially free of chemical precursors or other chemicals when chemically synthesized. The term embraces nucleic acid sequences that have been removed from their naturally occurring environment, and includes recombinant or cloned DNA isolates and chemically synthesized analogues or analogues biologically synthesized by heterologous systems. A substantially pure molecule may include isolated forms of the molecule.

“Polynucleotide” or “nucleic acid,” as used interchangeably herein, refers to polymers of nucleotides of any length and includes DNA and RNA. The nucleotides can be deoxyribonucleotides, ribonucleotides, modified nucleotides or bases, and/or their analogues, or any substrate that can be incorporated into a polymer by DNA or RNA polymerase or by a synthetic reaction. A polynucleotide may comprise modified nucleotides, such as methylated nucleotides and their analogues. “Oligonucleotide,” as used herein, refers to short, generally single-stranded, synthetic polynucleotides that are generally, but not necessarily, fewer than about 200 nucleotides in length. The terms “oligonucleotide” and “polynucleotide” are not mutually exclusive. The description above for polynucleotides is equally and fully applicable to oligonucleotides. Unless specified otherwise, the left-hand end of any single-stranded polynucleotide sequence disclosed herein is the 5′ end; the left-hand direction of double-stranded polynucleotide sequences is referred to as the 5′ direction. The direction of 5′ to 3′ addition of nascent RNA transcripts is referred to as the transcription direction; sequence regions on the DNA strand having the same sequence as the RNA transcript that are 5′ to the 5′ end of the RNA transcript are referred to as “upstream sequences”; sequence regions on the DNA strand having the same sequence as the RNA transcript that are 3′ to the 3′ end of the RNA transcript are referred to as “downstream sequences.”

The term “pharmaceutically acceptable” as used herein means being approved by a regulatory agency of the Federal or a state government, or listed in United States Pharmacopeia, European Pharmacopeia, or other generally recognized Pharmacopeia for use in animals, and more particularly in humans.

“Excipient” means a pharmaceutically-acceptable material, composition, or vehicle, such as a liquid or solid filler, diluent, solvent, or encapsulating material. Excipients include, for example, encapsulating materials or additives such as absorption accelerators, antioxidants, binders, buffers, carriers, coating agents, coloring agents, diluents, disintegrating agents, emulsifiers, extenders, fillers, flavoring agents, humectants, lubricants, perfumes, preservatives, propellants, releasing agents, sterilizing agents, sweeteners, solubilizers, wetting agents and mixtures thereof. The term “excipient” can also refer to a diluent, adjuvant (e.g., Freunds' adjuvant (complete or incomplete) or vehicle.

In some embodiments, excipients are pharmaceutically acceptable excipients. Examples of pharmaceutically acceptable excipients include buffers, such as phosphate, citrate, and other organic acids; antioxidants, including ascorbic acid; low molecular weight (e.g., fewer than about 10 amino acid residues) polypeptide; proteins, such as serum albumin, gelatin, or immunoglobulins; hydrophilic polymers, such as polyvinylpyrrolidone; amino acids, such as glycine, glutamine, asparagine, arginine, or lysine; monosaccharides, disaccharides, and other carbohydrates, including glucose, mannose, or dextrins; chelating agents, such as EDTA; sugar alcohols, such as mannitol or sorbitol; salt-forming counterions, such as sodium; and/or nonionic surfactants, such as TWEEN™, polyethylene glycol (PEG), and PLURONICS™. Other examples of pharmaceutically acceptable excipients are described in Remington and Gennaro, Remington's Pharmaceutical Sciences (18th ed. 1990).

In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio (See Williams & Wilkins eds., 2005, Handbook of Pharmaceutical Excipients, 6th ed.; Rowe et al., eds., 2009, Handbook of Pharmaceutical Additives, 3rd ed.; Ash and Ash eds., 2007, Pharmaceutical Preformulation and Formulation, 2nd ed). In some embodiments, pharmaceutically acceptable excipients are nontoxic to the cell or mammal being exposed thereto at the dosages and concentrations employed. In some embodiments, a pharmaceutically acceptable excipient is an aqueous pH buffered solution.

In some embodiments, excipients are sterile liquids, such as water and oils, including those of petroleum, animal, vegetable, or synthetic origin, such as peanut oil, soybean oil, mineral oil, sesame oil, and the like. Water is an exemplary excipient when a composition (e.g., a pharmaceutical composition) is administered intravenously. Saline solutions and aqueous dextrose and glycerol solutions can also be employed as liquid excipients, particularly for injectable solutions. An excipient can also include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol, and the like. The composition, if desired, can also contain minor amounts of wetting or emulsifying agents, or pH buffering agents. Compositions can take the form of solutions, suspensions, emulsion, tablets, pills, capsules, powders, sustained-release formulations, and the like. Oral compositions, including formulations, can include standard excipients such as pharmaceutical grades of mannitol, lactose, starch, magnesium stearate, sodium saccharine, cellulose, magnesium carbonate, etc.

Compositions, including pharmaceutical peptides, may contain a peptide, for example, in isolated or purified form, together with a suitable amount of excipients.

The term “effective amount” or “therapeutically effective amount” as used herein refers to the amount of a peptide or pharmaceutical composition provided herein which is sufficient to result in the desired outcome.

The terms “subject” and “patient” may be used interchangeably. As used herein, in certain embodiments, a subject is an animal, such as a non-primate (e.g., cow, pig, horse, cat, dog, rat, etc.) or a primate (e.g., monkey and human). In specific embodiments, the subject is a human. In one embodiment, the subject is a mammal, e.g., a human, diagnosed with a condition or disorder. In another embodiment, the subject is a mammal, e.g., a human, at risk of developing a condition or disorder.

“Administer” or “administration” refers to the act of injecting or otherwise physically delivering a substance as it exists outside the body into a patient, such as by mucosal, intradermal, intravenous, intramuscular delivery, and/or any other method of physical delivery described herein or known in the art.

As used herein, the terms “treat” “treatment” and “treating” refer to the reduction or amelioration of the progression, severity, and/or duration of a disease or condition resulting from the administration of one or more therapies. Treating may be determined by assessing whether there has been a decrease, alleviation and/or mitigation of one or more symptoms associated with the underlying disorder such that an improvement is observed with the patient, despite that the patient may still be afflicted with the underlying disorder. The term “treating” includes both managing and ameliorating the disease. The terms “manage,” “managing,” and “management” refer to the beneficial effects that a subject derives from a therapy which does not necessarily result in a cure of the disease.

The terms “prevent,” “preventing,” and “prevention” refer to reducing the likelihood of the onset (or recurrence) of a disease, disorder, condition, or associated symptom(s).

The terms “alleviate” and “alleviating” refer to easing or reducing one or more symptoms (e.g., pain) of a disorder, disease, or condition. The terms can also refer to reducing adverse effects associated with an active ingredient. Sometimes, the beneficial effects that a subject derives from a prophylactic or therapeutic agent do not result in a cure of the disorder, disease, or condition.

The term “contacting” or “contact” is meant to refer to bringing together of a therapeutic agent and cell or tissue such that a physiological and/or chemical effect takes place as a result of such contact. Contacting can take place in vitro, ex vivo, or in vivo. In one embodiment, a therapeutic agent is contacted with a cell in cell culture (in vitro) to determine the effect of the therapeutic agent on the cell. In another embodiment, the contacting of a therapeutic agent with a cell or tissue includes the administration of a therapeutic agent to a subject having the cell or tissue to be contacted.

In certain embodiments, the peptides described herein attenuates (e.g., partially attenuates) an amyloid β activity. In some embodiments, the peptides provided herein attenuate an amyloid β activity by at least about 10%. In some embodiments, the peptides provided herein attenuate an amyloid β activity by at least about 20%. In some embodiments, the peptides provided herein attenuate an amyloid β activity by at least about 30%. In some embodiments, the peptides provided herein attenuate an amyloid β activity at least about 40%. In some embodiments, the peptides provided herein attenuate an amyloid β activity by at least about 50%. In some embodiments, the peptides provided herein attenuate an amyloid β activity by at least about 60%. In some embodiments, the peptides provided herein attenuate an amyloid β activity by at least about 70%. In some embodiments, the peptides provided herein attenuate an amyloid activity by at least about 80%. In some embodiments, the peptides provided herein attenuate an amyloid β activity by at least about 90%. In some embodiments, the peptides provided herein attenuate an amyloid β activity by at least about 95%. In certain embodiments, the compounds described herein can attenuate (e.g., partially attenuate) an amyloid β activity by at least about 15% to about 65%. In certain embodiments, the compounds described herein can attenuate (e.g., partially attenuate) an amyloid β activity by at least about 30% to about 65%.

In specific embodiments, the attenuation of an amyloid β activity is assessed by methods known to one of skill in the art. In certain embodiments, the attenuation of an amyloid activity is relative to the amyloid β activity in the presence of stimulation without any of the compounds described herein.

A non-limiting example of an amyloid β activity is amyloid β-induced or -mediated signaling. Thus, in certain embodiments, the compound provided herein attenuates (e.g., partially attenuates) amyloid β-induced signaling. Another non-limiting example of amyloid β-induced signaling is interacting with (including blocking) receptors including but not limited to glucose transporters, NMDAR, AMPAR and acetylcholine receptors, activation of inflammatory signaling pathways, and the activation of one or more kinases including but not limited to GSK-3, CDK5, PKC, PKA and Erk1/2. Activities can include blocking ion channels, disruption of calcium homeostasis, mitochondrial oxidative stress, impaired energy metabolism, abnormal glucose regulation, and/or neuronal cell death.

In certain embodiments, the peptide described herein attenuates (e.g., partially attenuates) a tau protein activity. In some embodiments, the peptide provided herein attenuates a tau protein activity by at least about 10%. In some embodiments, the peptide provided herein attenuates a tau protein activity by at least about 20%. In some embodiments, the peptide provided herein attenuates a tau protein activity by at least about 30%. In some embodiments, the peptide provided herein attenuates a tau protein activity at least about 40%. In some embodiments, the peptide provided herein attenuates a tau protein activity by at least about 50%. In some embodiments, the peptide provided herein attenuates a tau protein activity by at least about 60%. In some embodiments, the peptide provided herein attenuates a tau protein activity by at least about 70%. In some embodiments, the peptide provided herein attenuates a tau protein activity by at least about 80%. In some embodiments, the peptide provided herein attenuates a tau protein activity by at least about 90%. In some embodiments, the peptide provided herein attenuates a tau protein activity by at least about 95%. In certain embodiments, the compounds described herein can attenuate (e.g., partially attenuate) a tau protein by at least about 15% to about 65%. In certain embodiments, the compounds described herein can attenuate (e.g., partially attenuate) a tau protein by at least about 30% to about 65%.

In specific embodiments, the attenuation of a tau protein activity is assessed by methods known to one of skill in the art. In certain embodiments, the attenuation of a tau protein activity is relative to the tau protein activity without any of the compounds described herein.

A non-limiting example of a tau protein activity is a tau protein-induced or -mediated signaling. Thus, in certain embodiments, the compound provided herein attenuates (e.g., partially attenuates) tau protein-induced signaling. Non-limiting examples of a tau protein activity include interacting with tubulin to stabilize microtubules, formation of helical and/or straight filaments, activation of inflammatory signaling pathways and impaired insulin signaling in the brain.

The term “about” or “approximately” means an acceptable error for a particular value as determined by one of ordinary skill in the art, which depends in part on how the value is measured or determined. In certain embodiments, the term “about” or “approximately” means within 1, 2, 3, or 4 standard deviations. In certain embodiments, the terms “about” and “approximately” mean within 20%, within 15%, within 10%, within 9%, within 8%, within 7%, within 6%, within 5%, within 4%, within 3%, within 2%, within 1%, or less of a given value or range.

The term “disease or disorder related to amyloid (or amyloid β),” “amyloid (or amyloid β) related disease or disorder,” “amyloid (or amyloid β) associated disease or disorder,” or a similar term, as used herein refers to any disease or disorder whose clinicopathological features include abnormal amount amyloid β (Aβ) or different isoforms of Aβ, including monomers, soluble oligomers, insoluble fibrils, and larger insoluble aggregates, such as Aβ plaques. The term “Aβ” or “amyloid β” are used interchangeably herein and refer to any products with varied lengths from cleaving β-APP by γ-secretase other than the intracellular domain of APP (AICD).

As used in the present disclosure and claims, the singular forms “a”, “an” and “the” include plural forms unless the context clearly dictates otherwise.

It is understood that wherever embodiments are described herein with the term “comprising” otherwise analogous embodiments described in terms of “consisting of” and/or “consisting essentially of” are also provided. It is also understood that wherever embodiments are described herein with the phrase “consisting essentially of” otherwise analogous embodiments described in terms of “consisting of” are also provided.

The term “between” as used in a phrase as such “between A and B” or “between A-B” refers to a range including both A and B.

The term “and/or” as used in a phrase such as “A and/or B” herein is intended to include both A and B; A or B; A (alone); and B (alone). Likewise, the term “and/or” as used in a phrase such as “A, B, and/or C” is intended to encompass each of the following embodiments: A, B, and C; A, B, or C; A or C; A or B; B or C; A and C; A and B; B and C; A (alone); B (alone); and C (alone).

### 5.2 the Peptides

We have previously disclosed a peptide comprising a sequence of DEEEDEEL (SEQ ID NO:1) (“P8”) that is identical to 66th-73rd amino acid segment of presenilin-1 that has β-APP binding activity (see SEQ ID NO: 5 in PCT Publication No. WO 10/132609). By binding to β-APP, said peptide prevents the full-length native presenilin-1 from binding to β-APP, thus acts in a dominant negative fashion to inhibit the biological activities mediated through binding of presenilin-1 and β-APP, thereby preventing the formation of Aβ.

Provided herein are novel peptides that are variants of P8 and exhibit unexpectedly superior stability and/or high efficiency of reducing Aβ levels. The peptides provided herein include, but are not limited to, synthetic peptides with C-terminal amidation, N-terminal acetylation, and/or partial substitution with D-amino acids.

More specifically, one peptide provided herein (named 8M1) comprises a sequence containing C-terminal amidation to SEQ ID NO:1 (i.e., amidation of the C-terminal amino acid Leu), and said peptide comprises the sequence Asp^{L}-Glu^{L}-Glu^{L}-Glu^{L}-Asp^{L}-Glu^{L}-Glu^{L}-Leu^{L}-CONH₂ or DEEEDEEL-NH₂ (SEQ ID NO:2).

Another peptide provided herein (named 8M2) comprises a sequence wherein an acetyl group is added to N terminus of SEQ ID NO: 2 (i.e., acetylation of the N-terminal amino acid Asp), and said peptide comprises the sequence CH₃CO—NH-Asp^{L}-Glu^{L}-Glu^{L}-Glu^{L}-Asp^{L}-Glu^{L}-Glu^{L}-Leu^{L}-CONH₂ or Ac-DEEEDEEL-NH₂ (SEQ ID NO: 3).

Yet another peptide provided herein (named 8M1D) comprises a sequence that substitutes the first amino acid of N terminus of SEQ ID NO: 2 (i.e., N-terminal amino acid Asp) with its D-amino acid, the enantiomer of L-amino acid, and said peptide comprises the sequence of Asp^{D}-Glu^{L}-Glu^{L}-Glu^{L}-Asp^{L}-Glu^{L}-Glu^{L}-Leu^{L}-CONH₂ or dEEEDEEL-NH₂ (SEQ ID NO:4).

Yet another peptide provided herein (named 8M2D) comprises a sequence that adds an acetyl group to the N terminus of SEQ ID NO: 4 (i.e., aceylation of the N-terminal amino acid Asp^{D}), and said peptide comprises the sequence CH₃CO—NH-Asp^{D}-Glu^{L}-Glu^{L}-Glu^{L}-Asp^{L}-Glu^{L}-Glu^{L}-Leu^{L}-CONH₂ or Ac-dEEEDEEL-NH₂ (SEQ ID NO:5).

In certain embodiments, the peptides provided herein attenuate the binding of beta-Amyloid precursor protein (β-APP) with presenilin-1 (PS-1) and/or presenilin-2 (PS-2) in a cell.

In some embodiments, the peptide provided herein attenuates (e.g., partially attenuates) the binding of beta-amyloid precursor protein (β-APP) with presenilin-1 (PS-1) and/or presenilin-2 (PS-2) in a cell by at least about 10%. In some embodiments, the peptide provided herein attenuates the binding of beta-amyloid precursor protein (β-APP) with presenilin-1 (PS-1) and/or presenilin-2 (PS-2) in a cell by at least about 20%. In some embodiments, the peptide provided herein attenuates the binding of beta-amyloid precursor protein (β-APP) with presenilin-1 (PS-1) and/or presenilin-2 (PS-2) in a cell by at least about 30%. In some embodiments, the peptide provided herein attenuates the binding of beta-amyloid precursor protein (β-APP) with presenilin-1 (PS-1) and/or presenilin-2 (PS-2) in a cell by at least about 40%. In some embodiments, the peptide provided herein attenuates the binding of beta-amyloid precursor protein (β-APP) with presenilin-1 (PS-1) and/or presenilin-2 (PS-2) in a cell by at least about 50%. In some embodiments, the peptide provided herein attenuates the binding of beta-amyloid precursor protein (β-APP) with presenilin-1 (PS-1) and/or presenilin-2 (PS-2) in a cell by at least about 60%. In some embodiments, the peptide provided herein attenuates the binding of beta-amyloid precursor protein (β-APP) with presenilin-1 (PS-1) and/or presenilin-2 (PS-2) in a cell by at least about 70%. In some embodiments, the peptide provided herein attenuates the binding of beta-amyloid precursor protein (β-APP) with presenilin-1 (PS-1) and/or presenilin-2 (PS-2) in a cell by at least about 80%. In some embodiments, the peptide provided herein attenuates the binding of beta-amyloid precursor protein (β-APP) with presenilin-1 (PS-1) and/or presenilin-2 (PS-2) in a cell by at least about 90%. In some embodiments, the peptide provided herein attenuates the binding of beta-amyloid precursor protein (β-APP) with presenilin-1 (PS-1) and/or presenilin-2 (PS-2) in a cell by at least about 95%.

In certain embodiments, the peptides provided herein attenuate (e.g., partially attenuate) the production of amyloid β. (Aβ) in a cell. In some embodiments, the peptide provided herein attenuates the production of Aβ in a cell by at least about 10%. In some embodiments, the peptide provided herein attenuates the production of Aβ in a cell by at least about 20%. In some embodiments, the peptide provided herein attenuates the production of Aβ in a cell by at least about 30%. In some embodiments, the peptide provided herein attenuates the production of Aβ in a cell by at least about 40%. In some embodiments, the peptide provided herein attenuates the production of Aβ in a cell by at least about 50%. In some embodiments, the peptide provided herein attenuates the production of Aβ in a cell by at least about 60%. In some embodiments, the peptide provided herein attenuates the production of Aβ in a cell by at least about 70%. In some embodiments, the peptide provided herein attenuates the production of Aβ in a cell by at least about 80%. In some embodiments, the peptide provided herein attenuates the production of Aβ in a cell by at least about 90%. In some embodiments, the peptide provided herein attenuates the production of Aβ in a cell by at least about 95%.

In certain embodiments, the peptides provided herein attenuate (e.g., partially attenuate) the production of amyloid β. (Aβ) in a subject. In some embodiments, the peptide provided herein attenuates the amount of Aβ in a subject's plasma by at least about 10%. In some embodiments, the peptide provided herein attenuates the amount of Aβ in a subject's plasma by at least about 20%. In some embodiments, the peptide provided herein attenuates the amount of Aβ in a subject's plasma by at least about 30%. In some embodiments, the peptide provided herein attenuates the amount of Aβ in a subject's plasma by at least about 40%. In some embodiments, the peptide provided herein attenuates the amount of Aβ in a subject's plasma by at least about 50%. In some embodiments, the peptide provided herein attenuates the amount of Aβ in a subject's plasma by at least about 60%. In some embodiments, the peptide provided herein attenuates the amount of Aβ in a subject's plasma by at least about 70%. In some embodiments, the peptide provided herein attenuates the amount of Aβ in a subject's plasma by at least about 80%. In some embodiments, the peptide provided herein attenuates the amount of Aβ in a subject's plasma by at least about 90%. In some embodiments, the peptide provided herein attenuates the amount of Aβ in a subject's plasma by at least about 95%.

In some embodiments, the peptide provided herein attenuates the amount of Aβ in a subject's CSF by at least about 10%. In some embodiments, the peptide provided herein attenuates the amount of Aβ in a subject's CSF by at least about 20%. In some embodiments, the peptide provided herein attenuates the amount of Aβ in a subject's CSF by at least about 30%. In some embodiments, the peptide provided herein attenuates the amount of Aβ in a subject's CSF by at least about 40%. In some embodiments, the peptide provided herein attenuates the amount of Aβ in a subject's CSF by at least about 50%. In some embodiments, the peptide provided herein attenuates the amount of Aβ in a subject's CSF by at least about 60%. In some embodiments, the peptide provided herein attenuates the amount of Aβ in a subject's CSF by at least about 70%. In some embodiments, the peptide provided herein attenuates the amount of Aβ in a subject's CSF by at least about 80%. In some embodiments, the peptide provided herein attenuates the amount of Aβ in a subject's CSF by at least about 90%. In some embodiments, the peptide provided herein attenuates the amount of Aβ in a subject's CSF by at least about 95%.

In certain embodiments, the peptides described herein attenuates (e.g., partially attenuates) an amyloid β activity. A non-limiting example of an amyloid β activity is amyloid β-induced or -mediated signaling. Thus, in certain embodiments, the peptides provided herein attenuates (e.g., partially attenuates) amyloid β-induced signaling. Another non-limiting example of amyloid β-induced signaling is interacting with (including blocking) receptors including but not limited to glucose transporters, NMDAR, AMPAR and acetylcholine receptors, activation of inflammatory signaling pathways, and the activation of one or more kinases including but not limited to GSK-3, CDK5, PKC, PKA and Erk1/2. Activities can include blocking ion channels, disruption of calcium homeostasis, mitochondrial oxidative stress, impaired energy metabolism, abnormal glucose regulation and/or neuronal cell death.

In some embodiments, the peptides provided herein attenuate an amyloid β activity (e.g., amyloid β induced signaling) in a cell by at least about 10%. In some embodiments, the peptides provided herein attenuate an amyloid β activity (e.g., amyloid β induced signaling) in a cell by at least about 20%. In some embodiments, the peptides provided herein attenuate an amyloid β activity (e.g., amyloid β induced signaling) in a cell by at least about 30%. In some embodiments, the peptides provided herein attenuate an amyloid β activity (e.g., amyloid β induced signaling) in a cell at least about 40%. In some embodiments, the peptides provided herein attenuate an amyloid β activity (e.g., amyloid β induced signaling) in a cell by at least about 50%. In some embodiments, the peptides provided herein attenuate an amyloid β activity (e.g., amyloid β induced signaling) in a cell by at least about 60%. In some embodiments, the peptides provided herein attenuate an amyloid β activity (e.g., amyloid β induced signaling) in a cell by at least about 70%. In some embodiments, the peptides provided herein attenuate an amyloid β activity (e.g., amyloid β induced signaling) in a cell by at least about 80%. In some embodiments, the peptides provided herein attenuate an amyloid β activity (e.g., amyloid β induced signaling) in a cell by at least about 90%. In some embodiments, the peptides provided herein attenuate an amyloid β activity (e.g., amyloid β induced signaling) in a cell by at least about 95%.

In some embodiments, the peptides provided herein attenuate an amyloid β activity (e.g., amyloid β induced signaling) in a subject by at least about 10%. In some embodiments, the peptides provided herein attenuate an amyloid β activity (e.g., amyloid β induced signaling) in a subject by at least about 20%. In some embodiments, the peptides provided herein attenuate an amyloid β activity (e.g., amyloid β induced signaling) in a subject by at least about 30%. In some embodiments, the peptides provided herein attenuate an amyloid β activity (e.g., amyloid β induced signaling) in a subject at least about 40%. In some embodiments, the peptides provided herein attenuate an amyloid β activity (e.g., amyloid β induced signaling) in a subject by at least about 50%. In some embodiments, the peptides provided herein attenuate an amyloid β activity (e.g., amyloid β induced signaling) in a subject by at least about 60%. In some embodiments, the peptides provided herein attenuate an amyloid β activity (e.g., amyloid β induced signaling) in a subject by at least about 70%. In some embodiments, the peptides provided herein attenuate an amyloid β activity (e.g., amyloid β induced signaling) in a subject by at least about 80%. In some embodiments, the peptides provided herein attenuate an amyloid β activity (e.g., amyloid β induced signaling) in a subject by at least about 90%. In some embodiments, the peptides provided herein attenuate an amyloid β activity (e.g., amyloid β induced signaling) in a subject by at least about 95%.

In certain embodiments, the peptides provided herein attenuate (e.g., partially attenuate) the production of Tau protein in a cell. In some embodiments, the peptide provided herein attenuates the production of Tau in a cell by at least about 10%. In some embodiments, the peptide provided herein attenuates the production of Tau in a cell by at least about 20%. In some embodiments, the peptide provided herein attenuates the production of Tau in a cell by at least about 30%. In some embodiments, the peptide provided herein attenuates the production of Tau in a cell by at least about 40%. In some embodiments, the peptide provided herein attenuates the production of Tau in a cell by at least about 50%. In some embodiments, the peptide provided herein attenuates the production of Tau in a cell by at least about 60%. In some embodiments, the peptide provided herein attenuates the production of Tau in a cell by at least about 70%. In some embodiments, the peptide provided herein attenuates the production of Tau in a cell by at least about 80%. In some embodiments, the peptide provided herein attenuates the production of Tau in a cell by at least about 90%. In some embodiments, the peptide provided herein attenuates the production of Tau in a cell by at least about 95%.

In certain embodiments, the peptides provided herein attenuate (e.g., partially attenuate) the production of Tau in a subject. In some embodiments, the peptide provided herein attenuates the amount of Tau in a subject's plasma by at least about 10%. In some embodiments, the peptide provided herein attenuates the amount of Tau in a subject's plasma by at least about 20%. In some embodiments, the peptide provided herein attenuates the amount of Tau in a subject's plasma by at least about 30%. In some embodiments, the peptide provided herein attenuates the amount of Tau in a subject's plasma by at least about 40%. In some embodiments, the peptide provided herein attenuates the amount of Tau in a subject's plasma by at least about 50%. In some embodiments, the peptide provided herein attenuates the amount of Tau in a subject's plasma by at least about 60%. In some embodiments, the peptide provided herein attenuates the amount of Tau in a subject's plasma by at least about 70%. In some embodiments, the peptide provided herein attenuates the amount of Tau in a subject's plasma by at least about 80%. In some embodiments, the peptide provided herein attenuates the amount of Tau in a subject's plasma by at least about 90%. In some embodiments, the peptide provided herein attenuates the amount of Tau in a subject's plasma by at least about 95%.

In certain embodiments, the peptide described herein attenuates (e.g., partially attenuates) a tau protein activity. A non-limiting example of a tau protein activity is a tau protein-induced or -mediated signaling. Thus, in certain embodiments, the peptides provided herein attenuates (e.g., partially attenuates) tau protein-induced signaling. Non-limiting examples of a tau protein activity include interacting with tubulin to stabilize microtubules, formation of helical and/or straight filaments, activation of inflammatory signaling pathways and impaired insulin signaling in the brain.

In some embodiments, the peptide provided herein attenuates a tau protein activity (e.g., tau protein-induced signaling) in a cell by at least about 10%. In some embodiments, the peptide provided herein attenuates a tau protein activity (e.g., tau protein-induced signaling) in a cell by at least about 20%. In some embodiments, the peptide provided herein attenuates a tau protein activity (e.g., tau protein-induced signaling) in a cell by at least about 30%. In some embodiments, the peptide provided herein attenuates a tau protein activity (e.g., tau protein-induced signaling) in a cell at least about 40%. In some embodiments, the peptide provided herein attenuates a tau protein activity (e.g., tau protein-induced signaling) in a cell by at least about 50%. In some embodiments, the peptide provided herein attenuates a tau protein activity (e.g., tau protein-induced signaling) in a cell by at least about 60%. In some embodiments, the peptide provided herein attenuates a tau protein activity (e.g., tau protein-induced signaling) in a cell by at least about 70%. In some embodiments, the peptide provided herein attenuates a tau protein activity (e.g., tau protein-induced signaling) in a cell by at least about 80%. In some embodiments, the peptide provided herein attenuates a tau protein activity (e.g., tau protein-induced signaling) in a cell by at least about 90%. In some embodiments, the peptide provided herein attenuates a tau protein activity (e.g., tau protein-induced signaling) in a cell by at least about 95%.

In some embodiments, the peptide provided herein attenuates a tau protein activity (e.g., tau protein-induced signaling) in a subject by at least about 10%. In some embodiments, the peptide provided herein attenuates a tau protein activity (e.g., tau protein-induced signaling) in a subject by at least about 20%. In some embodiments, the peptide provided herein attenuates a tau protein activity (e.g., tau protein-induced signaling) in a subject by at least about 30%. In some embodiments, the peptide provided herein attenuates a tau protein activity (e.g., tau protein-induced signaling) in a subject at least about 40%. In some embodiments, the peptide provided herein attenuates a tau protein activity (e.g., tau protein-induced signaling) in a subject by at least about 50%. In some embodiments, the peptide provided herein attenuates a tau protein activity (e.g., tau protein-induced signaling) in a subject by at least about 60%. In some embodiments, the peptide provided herein attenuates a tau protein activity (e.g., tau protein-induced signaling) in a subject by at least about 70%. In some embodiments, the peptide provided herein attenuates a tau protein activity (e.g., tau protein-induced signaling) in a subject by at least about 80%. In some embodiments, the peptide provided herein attenuates a tau protein activity (e.g., tau protein-induced signaling) in a subject by at least about 90%. In some embodiments, the peptide provided herein attenuates a tau protein activity (e.g., tau protein-induced signaling) in a subject by at least about 95%.

In some embodiments, the peptides provided herein include variants of the above-mentioned peptides.

In some embodiments, amino acid sequence modification(s) of the peptides provided herein are contemplated. For example, it may be desirable to modify or improve certain biological properties of the peptide, including but not limited to thermostability, expression level, glycosylation, and/or reduced immunogenicity. Thus, in addition to the peptides described herein, it is contemplated that peptide variants can be prepared. For example, peptide variants can be prepared by introducing appropriate nucleotide changes into the encoding DNA of the parental peptide, and/or by synthesis of the desired peptide.

Variations may be a substitution, deletion, or insertion of one or more codons encoding the parental peptide that results in a change in the amino acid sequence. Amino acid substitutions can be the result of replacing one amino acid with another amino acid having similar structural and/or chemical properties, such as the replacement of a leucine with a serine, e.g., conservative amino acid replacements. Standard techniques known to those of skill in the art can be used to introduce mutations in the nucleotide sequence encoding a molecule provided herein, including, for example, site-directed mutagenesis and PCR-mediated mutagenesis which results in amino acid substitutions. Substitutions, insertions or deletions may optionally be in the range of about 1 to 5 amino acids. In certain embodiments, the substitution, deletion, or insertion includes fewer than 10 amino acid substitutions, fewer than 5 amino acid substitutions, fewer than 4 amino acid substitutions, fewer than 3 amino acid substitutions, or fewer than 2 amino acid substitutions relative to the original molecule. In a specific embodiment, the substitution is a conservative amino acid substitution made at one or more predicted non-essential amino acid residues. The variation allowed may be determined by systematically making insertions, deletions, or substitutions of amino acids in the sequence and testing the resulting variants for activity exhibited by the full-length or mature native sequence.

A “conservative amino acid substitution” is one in which the amino acid residue is replaced with an amino acid residue having a side chain with a similar charge. Families of amino acid residues having side chains with similar charges have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine) and aromatic side chains (e.g., tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded protein can be expressed and the activity of the protein can be determined.

Amino acids may be grouped according to similarities in the properties of their side chains (see Lehninger, Biochemistry 73-75 (2d ed. 1975)): (1) non-polar: Ala (A), Val (V), Leu (L), Ile (I), Pro (P), Phe (F), Trp (W), Met (M); (2) uncharged polar: Gly (G), Ser (S), Thr (T), Cys (C), Tyr (Y), Asn (N), Gln (Q); (3) acidic: Asp (D), Glu (E); and (4) basic: Lys (K), Arg (R), His (H). Alternatively, naturally occurring residues may be divided into groups based on common side-chain properties: (1) hydrophobic: Norleucine, Met, Ala, Val, Leu, Ile; (2) neutral hydrophilic: Cys, Ser, Thr, Asn, Gln; (3) acidic: Asp, Glu; (4) basic: His, Lys, Arg; (5) residues that influence chain orientation: Gly, Pro; and (6) aromatic: Trp, Tyr, Phe.

In one embodiment, a peptide provided herein comprises an amino acid sequence that is at least 70%, at least 75%, at least 80%, at least 85%, at least 90%, at least 95%, or at least 99% identical to the amino acid sequence of the peptides described in the example section below.

The variations can be made using methods known in the art such as oligonucleotide-mediated (site-directed) mutagenesis, alanine scanning, and PCR mutagenesis. Site-directed mutagenesis (see Carter, 1986, Biochem J. 237:1-7; and Zoller et al., 1982, Nucl. Acids Res. 10:6487-500), cassette mutagenesis (see Wells et al., 1985, Gene 34:315-23), or other known techniques can be performed on the cloned DNA to produce variant DNA of a parental peptide.

Encompassed by the disclosure are oligomers or fusion polypeptides that comprise peptides provided herein. In some embodiments, the oligomers comprise peptides provided herein. Oligomers can be in the form of covalently linked or non-covalently-linked multimers, including dimers, trimers, or higher oligomers. In some embodiments, the oligomers maintain the binding ability of the polypeptide components and provide therefore, bivalent, trivalent, etc., binding sites. In some embodiment the disclosure is directed to oligomers comprising multiple peptides provided herein joined via covalent or non-covalent interactions between peptide moieties fused to the polypeptides, such peptides having the property of promoting oligomerization.

The present disclosure also provides conjugates comprising any one of the peptides of the present disclosure covalently bound by a synthetic linker to one or more agents.

In some embodiments, the peptides provided herein are conjugated or recombinantly fused, e.g., to another therapeutic agent (e.g., a cytotoxic agent) or a diagnostic or detectable molecule. The conjugated or recombinantly fused peptide can be useful, for example, for treating or preventing a disease or disorder. The conjugated or recombinantly fused peptide can be useful, for example, for monitoring or prognosing the onset, development, progression, and/or severity of a disease or disorder.

Such diagnosis and detection can be accomplished, for example, by coupling the peptide to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidin/biotin or avidin/biotin; fluorescent materials, such as, but not limited to, umbelliferone, fluorescein, fluorescein isothiocynate, rhodamine, dichlorotriazinylamine fluorescein, dansyl chloride, or phycoerythrin; luminescent materials, such as, but not limited to, luminol; bioluminescent materials, such as, but not limited to, luciferase, luciferin, or aequorin; chemiluminescent material, such as, but not limited to, an acridinium based compound or a HALOTAG; radioactive materials, such as, but not limited to, iodine (131I, 125I, 123I, and 121I), carbon (14C), sulfur (35S), tritium (3H), indium (115In, 113In, 112In, and 111In), technetium (99Tc), thallium (201Ti), gallium (68Ga and 67Ga), palladium (103Pd), molybdenum (99Mo), xenon (133Xe), fluorine (18F), 153Sm, 177Lu, 159Gd, 149Pm, 140La, 175Yb, 166Ho, 90Y, 47Sc, 186Re, 188Re, 142Pr, 105Rh, 97Ru, 68Ge, 57Co, 65Zn, 85Sr, 32P, 153Gd, 169Yb, 51Cr, 54Mn, 75Se, 113Sn, or 117Sn; positron emitting metals using various positron emission tomographies; and non-radioactive paramagnetic metal ions.

Also provided herein are peptides that are recombinantly fused or chemically conjugated (covalent or non-covalent conjugations) to a heterologous protein or polypeptide (or fragment thereof, for example, to a polypeptide of about 10, about 20, about 30, about 40, about 50, about 60, about 70, about 80, about 90, or about 100 amino acids) to generate fusion proteins, as well as uses thereof. In particular, provided herein are fusion proteins comprising a peptide provided herein and a heterologous protein, polypeptide, or peptide. In one embodiment, the heterologous protein, polypeptide, or peptide that the peptide provided herein is fused to is useful for targeting the peptide to a particular cell type.

Encompassed by the disclosure are immunoglobulin-based oligomers. The peptides provided herein may be fused to molecules such as immunoglobulins for many purposes, including increasing the valency of polypeptide binding sites. For example, the peptides provided herein may be fused directly or through a linker peptide to the Fc portion of an immunoglobulin, wherein the immunoglobulin can be IgG molecule, or other isotypes, such as IgM molecule. The term “Fc polypeptide” as used herein includes native and mutant forms of polypeptides made up of the Fc region of an antibody comprising any or all of the CH domains of the Fc region.

Moreover, peptides provided herein can be fused to marker or “tag” sequences, such as a peptide, to facilitate purification. In specific embodiments, the marker or tag amino acid sequence is a hexa-histidine peptide, such as the tag provided in a pQE vector (see, e.g., QIAGEN, Inc.), among others, many of which are commercially available. For example, as described in Gentz et al., 1989, Proc. Natl. Acad. Sci. USA 86:821-24, hexa-histidine provides for convenient purification of the fusion protein. Other peptide tags useful for purification include, but are not limited to, the hemagglutinin (“HA”) tag, which corresponds to an epitope derived from the influenza hemagglutinin protein (see Wilson et al., 1984, Cell 37:767-78), and the “FLAG” tag.

Methods for fusing or conjugating moieties (including polypeptides) to peptides are known in the art. Fusion proteins may be generated, for example, through the techniques of gene-shuffling, motif-shuffling, exon-shuffling, and/or codon-shuffling (collectively referred to as “DNA shuffling”). Peptides provided herein may be altered by being subjected to random mutagenesis by error-prone PCR, random nucleotide insertion, or other methods prior to recombination. A polynucleotide encoding a peptide provided herein may be recombined with one or more components, motifs, sections, parts, domains, fragments, etc. of one or more heterologous molecules.

Peptides as provided herein may also be attached to solid supports, which are particularly useful for immunoassays or purification of the binding partner. Such solid supports include, but are not limited to, glass, cellulose, polyacrylamide, nylon, polystyrene, polyvinyl chloride, or polypropylene.

The linker may be a “cleavable linker” facilitating release of the conjugated agent in the cell, but non-cleavable linkers are also contemplated herein. Linkers for use in the conjugates of the present disclosure include, without limitation, acid labile linkers (e.g., hydrazone linkers), disulfide-containing linkers, peptidase-sensitive linkers (e.g., peptide linkers comprising amino acids, for example, valine and/or citrulline such as citrulline-valine or phenylalanine-lysine), photolabile linkers, dimethyl linkers (see Chari et al., 1992, Cancer Res. 52:127-31; and U.S. Pat. No. 5,208,020), thioether linkers, or hydrophilic linkers designed to evade multidrug transporter-mediated resistance (see Kovtun et al., 2010, Cancer Res. 70:2528-37).

Conjugates of the peptide and agent may be made using a variety of bifunctional protein coupling agents such as BMPS, EMCS, GMBS, HBVS, LC-SMCC, MBS, MPBH, SBAβ, SIA, SIAB, SMCC, SMPB, SMPH, sulfo-EMCS, sulfo-GMBS, sulfo-KMUS, sulfo-MBS, sulfo-SIAB, sulfo-SMCC, sulfo-SMPB, and SVSB (succinimidyl-(4-vinylsulfone)benzoate). The present disclosure further contemplates that conjugates of peptide and agents may be prepared using any suitable methods as disclosed in the art (see Hermanson eds., 2008, Bioconjugate Techniques 2d ed.).

### 5.3 Pharmaceutical Compositions

In one aspect, the present disclosure further provides pharmaceutical compositions comprising at least one peptide of the present disclosure. In some embodiments, a pharmaceutical composition comprises therapeutically effective amount of a peptide provided herein and a pharmaceutically acceptable excipient.

Pharmaceutical compositions comprising a peptide are prepared for storage by mixing the fusion protein having the desired degree of purity with optional physiologically acceptable excipients (see Remington, 1980, Remington's Pharmaceutical Sciences 18th ed.) in the form of aqueous solutions or lyophilized or other dried forms.

The peptide of the present disclosure may be formulated in any suitable form for delivery to a target cell/tissue, e.g., as microcapsules or macroemulsions (Remington, supra; see Park et al., 2005, Molecules 10:146-61; Malik et al., 2007, Curr. Drug. Deliv. 4:141-51), as sustained release formulations (see Putney and Burke, 1998, Nature Biotechnol. 16:153-57), or in liposomes (see Maclean et al., 1997, Int. J. Oncol. 11:325-32; Kontermann, 2006, Curr. Opin. Mol. Ther. 8:39-45).

A peptide provided herein can also be entrapped in microcapsule prepared, for example, by coacervation techniques or by interfacial polymerization, for example, hydroxymethylcellulose or gelatin-microcapsule and poly-(methylmethacylate) microcapsule, respectively, in colloidal drug delivery systems (for example, liposomes, albumin microspheres, microemulsions, nano-particles, and nanocapsules) or in macroemulsions. Such techniques are disclosed, for example, in Remington, supra.

Various compositions and delivery systems are known and can be used with a peptide as described herein, including, but not limited to, encapsulation in liposomes, microparticles, microcapsules, recombinant cells capable of expressing the peptide, receptor-mediated endocytosis (see Wu and Wu, 1987, J. Biol. Chem. 262:4429-32), construction of a nucleic acid as part of a retroviral or other vector, etc. In another embodiment, a composition can be provided as a controlled release or sustained release system. In one embodiment, a pump may be used to achieve controlled or sustained release (see Langer, supra; Sefton, 1987, Crit. Ref. Biomed. Eng. 14:201-40; Buchwald et al., 1980, Surgery 88:507-16; and Saudek et al., 1989, N. Engl. J. Med. 321:569-74). In another embodiment, polymeric materials can be used to achieve controlled or sustained release of a prophylactic or therapeutic agent (e.g., a peptide as described herein) or a composition provided herein (see Langer and Wise eds., 1974, Medical Applications of Controlled Release; Smolen and Ball eds., 1984, Controlled Drug Bioavailability, Drug Product Design and Performance; Ranger and Peppas, 1983, J. Macromol. Sci. Rev. Macromol. Chem. 23:61-126; Levy et al., 1985, Science 228:190-92; During et al., 1989, Ann. Neurol. 25:351-56; Howard et al., 1989, J. Neurosurg. 71:105-12; U.S. Pat. Nos. 5,679,377; 5,916,597; 5,912,015; 5,989,463; and 5,128,326; PCT Publication Nos. WO 99/15154 and WO 99/20253). Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In one embodiment, the polymer used in a sustained release formulation is inert, free of leachable impurities, stable on storage, sterile, and biodegradable.

In yet another embodiment, a controlled or sustained release system can be placed in proximity of a particular target tissue, for example, the nasal passages or lungs, thus requiring only a fraction of the systemic dose (see Goodson, 1984, Medical Applications of Controlled Release Vol. 2, 115-38). Controlled release systems are discussed, for example, by Langer, 1990, Science 249:1527-33. Any technique known to one of skill in the art can be used to produce sustained release formulations comprising one or more peptide as described herein (see U.S. Pat. No. 4,526,938, PCT publication Nos. WO 91/05548 and WO 96/20698, Ning et al., 1996, Radiotherapy & Oncology 39:179-89; Song et al., 1995, PDA J. of Pharma. Sci. & Tech. 50:372-97; Cleek et al., 1997, Pro. Int'l. Symp. Control. Rel. Bioact. Mater. 24:853-54; and Lam et al., 1997, Proc. Int'l. Symp. Control Rel. Bioact. Mater. 24:759-60).

### 5.4 Method of Making the Peptides

In yet another aspect, provided herein are methods for making the various peptides provided herein. The peptides provided herein can be made by various chemical synthesis methods, recombinant methods, or combinations thereof.

A peptide provided herein may be produced by known conventional chemical synthesis. Methods for constructing peptides of the disclosure by synthetic means are known to those skilled in the art. The synthetically constructed peptides, by virtue of sharing primary, secondary, or tertiary structural and/or conformational characteristics with native peptides may possess biological properties in common therewith, including peptide activity.

In some embodiments, the peptides provided herein may be prepared using conventional step-wise solution or solid phase synthesis (see Merrifield, R. B., 1963, J. Am. Chem. Soc. 85:2149-2154; Williams et al., Eds., 1997, Chemical Approaches to the Synthesis of Peptides and Proteins and references cited therein; Atherton & Sheppard, Eds., 1989, Solid Phase Peptide Synthesis: A Practical Approach and references cited therein).

Alternatively, the peptides provided herein may be prepared by way of segment condensation, as described, for example, in Liu et al., 1996, Tetrahedron Lett. 37(7):933-936; Baca, et al., 1995, J. Am. Chem. Soc. 117:1881-1887; Tam et al., 1995, Int. J. Peptide Protein Res. 45:209-216; Schnolzer and Kent, 1992, Science 256:221-225; Liu and Tam, 1994, J. Am. Chem. Soc. 116(10):4149-4153; Liu and Tam, 1994, Proc. Natl. Acad. Sci. USA 91:6584-6588; Yamashiro and Li, 1988, Int. J. Peptide Protein Res. 31:322-334). This is particularly the case with Gly (G) containing peptides. Other methods useful for synthesizing the peptides and peptide analogues of the invention are described in Nakagawa et al., 1985, J. Am. Chem. Soc. 107:7087-7092.

The peptides provided herein may be synthesized with one or more (D)-amino acids. The choice of including an (L)- or (D)-amino acid into a peptide depends, in part, upon the desired characteristics of the peptide. Replacement of all or part of a sequence of (L)-amino acids by the respective sequence of entatiomeric (D)-amino acids renders an optically isomeric structure in the respective part of the peptide chain. Inversion of the sequence of all or part of a sequence of (L)-amino acids renders retro-analogues of the peptide. Combination of the enantiomeric (L to D, or D to L) replacement and inversion of the sequence renders retro-inverso-analogues of the peptide. It is known to those skilled in the art that enantiomeric peptides, their retro-analogues, and their retro-inverso-analogues may maintain significant topological relationship to the parent peptide, and especially high degree of resemblance is often obtained for the parent and its retro-inverso-analogues. This relationship and resemblance can be reflected in biochemical properties of the peptides, especially high degrees of binding of the respective peptides and analogues to a receptor protein. The synthesis of the properties of retro-inverso anologues of peptides have been discussed for example in Methods of Organic Chemistry (Houben-Weyl), Synthesis of Peptides and Peptidomimetics—Workbench Edition Volume E22c (Editor-in-chief Goodman M.) 2004 (George Thieme Verlag Stuttgart, New York), and in references cited therein, all of which are hereby incorporated by reference herein in their entireties.

Amino acid modification includes the alteration of a naturally occurring amino acid to produce a non-naturally occurring amino acid. Derivatives of the peptides of the present invention with non-naturally occurring amino acids can be created by chemical synthesis or by site specific incorporation of unnatural amino acids into peptides during biosynthesis, as described in Christopher J. Noren, Spencer J. Anthony-Cahill, Michael C. Griffith, Peter G. Schultz, 1989 Science, 244:182-188, hereby incorporated by reference herein in its entirety.

Peptide mimetics that are structurally similar to therapeutically useful peptides may be used to produce an equivalent therapeutic or prophylactic effect. Generally, peptidomimetics are structurally similar to a paradigm polypeptide (i.e., a polypeptide that has a biochemical property or pharmacological activity), but have one or more peptide linkages optionally replaced by a linkage selected from the group consisting of: —CH2-NH—, —CH2S—, —CH2-CH2-, —CH═CH— (cis and trans), —COCH2-, —CH(OH)CH2-, and —CH2SO—, by methods known in the art and further described in the following references: Spatola, 1983, Peptide Backbone Modifications; Morely, 1098, Trends Pharma Sci, pp. 463-468; Hudson et al., 1979, Int J Pept Prot Re 14: 177-185 (—CH2-NH—, —CH2-CH2-); Spatola. et al., 1986, Life Sci 38:1243-1249 (—CH2-S—); Hann, 1982, J. Chem. Soc. Perkin. Trans. I 307-314 (—CH═CH—, cis and trans); Almquist et al., 1980, J. Med. Chem. 23: 1392 (—COCH2-); Jennings-White et al., 1982, Tetrahedron Lett 23:2533 (—COCH2-); Szelke et al., 1982, European Appln. EP 45665 CA: 97: 39405 (—CH(OH)CH2-); Holladay et al., 1983, Tetrahedron Lett 24:4401-4404 (—C(OH)CH2-); and Hruby, 1982, Life Sci 31:189-199 (—CH2-S—); each of which is incorporated herein by reference.

In another embodiment, a particularly preferred non-peptide linkage is —CH2NH—. Such peptide mimetics may have significant advantages over peptide embodiments, including, for example, more economical production, greater chemical stability, enhanced pharmacological properties (half-life, absorption, potency, efficacy, etc.), altered specificity (e.g., a broad-spectrum of biological activities), reduced antigenicity, and others.

A variety of designs for peptide mimetics are possible. For example, cyclic peptides, in which the necessary conformation is stabilized by non-peptides, are specifically contemplated, U.S. Pat. No. 5,192,746 to U.S. Pat. No. 5,576,423 to U.S. Pat. Nos. 5,051,448, and 5,559,103, all hereby incorporated by reference, describe multiple methods for creating such peptides. Synthesis of nonpeptide peptides that mimic peptide sequences is also known in the art. (see Eldred et al., 1994, J. Med. Chem. 37:3882, hereby incorporated by reference herein in its entirety) describe non-peptide antagonists that mimic the peptide sequence. Likewise, it is further elucidated the synthesis of a series of such peptides (see Ku et al., 1995, J. Med. Chem 38:9, hereby incorporated by reference herein in its entirety)

Further modifications following synthesis may be implemented. For example, the peptides may be further chemically modified, i.e. carbamylated, acetylated, succinylated, guanidated, nitrated, trinitrophenylated, amidinated, etc., in accordance with U.S. patent application Ser. No. 10/188,905, which published as 20030072737-A1 on Apr. 17, 2003 and discloses chemically modified EPO, and in accordance with U.S. patent application Ser. No. 10/612,665, filed Jul. 1, 2003, and U.S. patent application Ser. No. 09/753,132, filed Dec. 29, 2000, which are incorporated by reference herein in their entirety.

Additionally, the peptides may consist of recombinant peptides—muteins. The disclosed mutations may include substitutions, deletions, including internal deletions, additions, including additions yielding fusion proteins, or conservative substitutions of amino acid residues within and/or adjacent to the amino acid sequence, but that result in a “silent” change, and non-conservative amino acid changes and larger insertions and deletions, as previously disclosed in PCT/US03/20964 entitled Recombinant Tissue Protective Cytokines and Encoding Nucleic Acids Thereof for Protection, Restoration, and Enhancement of Responsive Cells, Tissues, and Organs (which is incorporated by reference herein in its entirety)

Either conservative or non-conservative amino acid substitutions can be made at one or more amino acid residues. Both conservative and non-conservative substitutions can be made. Conservative replacements are those that take place within a family of amino acids that are related in their side chains. Genetically encoded amino acids can be divided into four families: (1) acidic=Asp (D), Glu (G); (2) basic=Lys (K), Arg (R), His (H); (3) nonpolar (hydrophobic)=Cys (C), Ala (A), Val (V), Leu (L), Ile (I), Pro (P), Phe (F), Met (M), Trp (W), Gly (G), Tyr (Y); and (4) uncharged polar=Asn (N), Gln (Q), Ser (S), Thr (T). Non-polar may be subdivided into: strongly hydrophobic=Ala (A), Val (V), Leu (L), Ile (I), Met (M), Phe (F); and moderately hydrophobic=Gly (G), Pro (P), Cys (C), Tyr (Y), Trp (W). In alternative fashion, the amino acid repertoire can be grouped as (1) acidic=Asp (D), Glu (G); (2) basic=Lys (K), Arg (R), His (H), (3) aliphatic=Gly (G), Ala (A), Val (V), Leu (L), Ile (I), Ser (S), Thr (T), with Ser (S) and Thr (T) optionally be grouped separately as aliphatic-hydroxyl; (4) aromatic=Phe (F), Tyr (Y), Trp (W); (5) amide=Asn (N), Glu (Q); and (6) sulfur-containing=Cys (C) and Met (M). (See Stryer and Freeman eds., 1995, Biochemistry, 4th ed hereby incorporated by reference herein in its entirety).

Alternatively, mutations can be introduced randomly along all or part of the coding sequence of a peptide, such as by saturation mutagenesis, and the resultant mutants can be screened for biological activity to identify mutants that retain activity. Following mutagenesis, the encoded peptide can be expressed recombinantly and the activity of the recombinant peptide can be determined.

In another embodiment, the peptide may be further modified through the additions of polymers (such as polyethylene glycol), sugars, or additional proteins (such as a fusion construct) in an effort to extend the half-life of the peptide or enhance the peptide's tissue protective effects. Examples of such modifications are disclosed within WO/04022577 A3 and WO/05025606 A1, which are incorporated herein by reference.

Depending on the conjugation chemistry selected and the number of reactive sites already present or created on the peptide, one, two, or a selected number of polymers can be appended in a reproducible manner. The principal mode of attachment of a PEG, and its derivatives, to peptides is a non-specific bonding through a peptide amino acid residue (see U.S. Pat. Nos. 4,088,538, 4,496,689, 4,414,147, 4,055,635, and PCT WO 87/00056). Another mode of attaching PEG to peptides is through the non-specific oxidation of glycosyl residues on a glycopeptide (see WO 94/05332). In these non-specific methods, PEG is added in a random, non-specific manner to reactive residues on a peptide backbone.

In certain embodiments, the peptide provided herein can be made by recombinantly producing a parental peptide and then chemically modifying the parental peptide. For example, certain peptides provided herein can be produced by chemically modifying recombinantly produced P8 peptide described in the example section below.

Producing a peptide by a recombinant method is known in the art. More specifically, polynucleotides can be identified in several ways, including isolation of genomic or cDNA molecules from a suitable source. Nucleotide sequences corresponding to the amino acid sequences described herein, to be used as probes or primers for the isolation of polynucleotides or as query sequences for database searches, can be obtained by “back-translation” from the amino acid sequences, or by identification of regions of amino acid identity with peptides for which the coding DNA sequence has been identified. The well-known polymerase chain reaction (PCR) procedure can be employed to isolate and amplify a DNA sequence encoding a human Presenilin-1. Oligonucleotides that define the desired termini of the combination of DNA fragments are employed as 5′ and 3′ primers. The oligonucleotides can additionally contain recognition sites for restriction endonucleases, to facilitate insertion of the amplified combination of DNA fragments into an expression vector. PCR techniques are described in Saiki et al., 1988, Science 239:487; Wu et al., eds., 1989, Recombinant DNA Methodology pp. 189-196; and, Innis et al., eds., 1990, PCR Protocols: A Guide to Methods and Applications.

Polynucleotide molecules of the disclosure (e.g., polynucleotide of P8) include DNA and RNA in both single-stranded and double-stranded form, as well as the corresponding complementary sequences. DNA includes, for example, cDNA, genomic DNA, chemically synthesized DNA, DNA amplified by PCR, and combinations thereof. The polynucleotide molecules of the disclosure include full-length genes or cDNA molecules as well as a combination of fragments thereof. The polynucleotides of the disclosure can be derived from human sources, but the disclosure includes those derived from non-human species, as well.

An “isolated polynucleotide” is a polynucleotide that has been separated from adjacent genetic sequences present in the genome of the organism from which the polynucleotide was isolated, in the case of polynucleotides isolated from naturally occurring sources. In the case of polynucleotides synthesized enzymatically from a template or chemically, such as PCR products, cDNA molecules, or oligonucleotides for example, it is understood that the polynucleotides resulting from such processes are isolated polynucleotides. An isolated polynucleotide refers to a polynucleotide in the form of a separate fragment or as a component of a larger polynucleotide construct. In one embodiment, the disclosure relates to certain isolated polynucleotides that are substantially free from contaminating endogenous material. The polynucleotide has preferably been derived from DNA or RNA isolated at least once in substantially pure form and in a quantity or concentration enabling identification, manipulation, and recovery of its component nucleotide sequences by standard biochemical methods (see Sambrook et al., 1989, Molecular Cloning. A Laboratory Manual, 2nd ed). Such sequences are typically provided and/or constructed in the form of an open reading frame uninterrupted by internal non-translated sequences, or intrans, that are typically present in eukaryotic genes. Sequences of non-translated DNA can be present 5′ or 3′ from an open reading frame, where the same do not interfere with manipulation or expression of the coding region.

Expression, isolation, and purification of the parental peptide can be accomplished by any suitable technique, including but not limited to, the following methods. The isolated nucleic acid of the disclosure can be operably linked to an expression control sequence such as the pDC409 vector (Giri et al., 1990, EMBO J. 13: 2821) or the derivative pDC412 vector (Wiley et al., 1995, Immunity 3: 673). The pDC400 series vectors are useful for transient mammalian expression systems, such as CV-1 or 293 cells. Alternatively, the isolated nucleic acid of the disclosure can be linked to expression vectors such as pDC312, pDC316, or pDC317 vectors. The pDC300 series vectors all contain the SV40 origin of replication, the CMV promoter, the adenovirus tripartite leader, and the SV40 polyA and termination signals, and are useful for stable mammalian expression systems, such as CHO cells or their derivatives. Other expression control sequences and cloning technologies can also be used to produce the peptide recombinantly, such as the pMT2 or pED expression vectors (Kaufman et al., 1991, Nucleic Acids Res 19: 4485-4490; and Pouwels et al., 1985, Cloning Vectors. A Laboratory Manual) and the GATEWAY Vectors (Life Technologies; Rockville, Md.). The isolated nucleic acid of the disclosure, flanked by attB sequences, can be recombined through an integrase reaction with a GATEWAY vector such as pDONR201 containing attP sequences, providing an entry vector for the GATEWAY system containing the isolated nucleic acid of the disclosure. This entry vector can be further recombined with other suitably prepared expression control sequences, such as those of the pDC400 and pDC300 series described above. Many suitable expression control sequences are known in the art. General methods of expressing recombinant peptides are also described in Kaufman, 1990, Methods in Enzymology 185, 537-566. As used herein “operably linked” means that a polynucleotide of the disclosure and an expression control sequence are situated within a construct, vector, or cell in such a way that a peptide encoded by a polynucleotide is expressed when appropriate molecules (such as polymerases) are present. As one embodiment of the disclosure, at least one expression control sequence is operably linked to a polynucleotide of the disclosure in a recombinant host cell or progeny thereof, the polynucleotide and/or expression control sequence having been introduced into the host cell by transformation or transfection, for example, or by any other suitable method. As another embodiment of the disclosure, at least one expression control sequence is integrated into the genome of a recombinant host cell such that it is operably linked to a polynucleotide sequence encoding a peptide of the disclosure. In a further embodiment of the disclosure, at least one expression control sequence is operably linked to a polynucleotide of the disclosure through the action of a trans-acting factor such as a transcription factor, either in vitro or in a recombinant host cell.

In addition, a sequence encoding an appropriate signal peptide (native or heterologous) can be incorporated into expression vectors. The choice of signal peptide or leader can depend on factors such as the type of host cells in which the recombinant peptide is to be produced. To illustrate, examples of heterologous signal peptides that are functional in mammalian host cells include the signal sequence for interleukin-7 (IL-7) described in U.S. Pat. No. 4,965,195; the signal sequence for interleukin-2 receptor described in Cosman et al., 1984, Nature 312:768; the interleukin-4 receptor signal peptide described in EP 367,566; the type I interleukin-1 receptor signal peptide described in U.S. Pat. No. 4,968,607; and the type II interleukin-1 receptor signal peptide described in EP 460,846. A DNA sequence for a signal peptide (secretory leader) can be fused in frame to a polynucleotide of the disclosure so that the DNA is initially transcribed, and the mRNA translated, into a fusion peptide comprising the signal peptide. A signal peptide that is functional in the intended host cells promotes extracellular secretion of the peptide. The signal peptide is cleaved from the peptide upon secretion of peptide from the cell. The skilled artisan will also recognize that the position(s) at which the signal peptide is cleaved can differ from that predicted by computer program, and can vary according to such factors as the type of host cells employed in expressing a recombinant peptide. A peptide preparation can include a mixture of peptide molecules having different N-terminal amino acids, resulting from cleavage of the signal peptide at more than one site.

Established methods for introducing DNA into mammalian cells have been described (Kaufman, 1990, Large Scale Mammalian Cell Culture, pp. 15-69). Additional protocols using commercially available reagents, such as Lipofectamine lipid reagent (Gibco/BRL) or Lipofectamine-Plus lipid reagent, can be used to transfect cells (see Felgner et al., 1987, Proc. Natl. Acad. Sci. USA 84: 7413-7417). In addition, electroporation can be used to transfect mammalian cells using conventional procedures, such as those in Sambrook et al. 1989, Molecular Cloning: A Laboratory Manual, 2ed. Selection of stable transformants can be performed using methods known in the art such as, for example, resistance to cytotoxic drugs. Kaufman et al., 1990, Meth. in Enzymology 185:487-511 describes several selection schemes, such as dihydrofolate reductase (DHFR) resistance. A suitable strain for DHFR selection can be CHO strain DX-B11, which is deficient in DHFR (Urlaub and Chasin, Proc. Natl. Acad. Sci. USA 77:4216-4220, 1980). A plasmid expressing the DHFR cDNA can be introduced into strain DX-B11, and only cells that contain the plasmid can grow in the appropriate selective media. Other examples of selectable markers that can be incorporated into an expression vector include cDNAs conferring resistance to antibiotics, such as G418 and hygromycin B. Cells harboring the vector can be selected on the basis of resistance to these compounds.

Alternatively, gene products can be obtained via homologous recombination, or “gene targeting,” techniques. Such techniques employ the introduction of exogenous transcription control elements (such as the CMV promoter or the like) in a particular predetermined site on the genome, to induce expression of the endogenous polynucleotide sequence of interest. The location of integration into a host chromosome or genome can be easily determined by one of skill in the art, given the known location and sequence of the gene. In one embodiment, the disclosure also contemplates the introduction of exogenous transcriptional control elements in conjunction with an amplifiable gene, to produce increased amounts of the gene product, again, without the need for isolation of the gene itself from the host cell. The practice of homologous recombination or gene targeting is explained by Schimke, et al., 1987, Methods in Enzymology 151:85-104, as well as by Capecchi, et al., 1989, TIG 5:70-76.

A number of types of cells may act as suitable host cells for expression of a peptide. Mammalian host cells include, for example, the COS-7 line of monkey kidney cells (ATCC CRL 1651; see Gluzman et al., 1981, Cell 23:175), L cells, C127 cells, 3T3 cells (ATCC CCL 163), Chinese hamster ovary (CHO) cells, HeLa cells, BHK (ATCC CRL 10) cell lines, the CV1/EBNA cell line derived from the African green monkey kidney cell line CV1 (ATCC CCL 70) as described by McMahan et al., 1991, EMBO J. 10: 2821, human kidney 293 cells, human epidermal A431 cells, human Colo205 cells, other transformed primate cell lines, normal diploid cells, cell strains derived from in vitro culture of primary tissue, primary explants, HL-60, U937, HaK or Jurkat cells. Alternatively, it may be possible to produce a peptide in lower eukaryotes such as yeast or in prokaryotes such as bacteria. Potentially suitable yeast strains include Saccharomyces cerevisiae, Schizosaccharomyces pombe, Kluyveromyces strains, Candida, or any yeast strain capable of expressing heterologous peptides. Potentially suitable bacterial strains include Escherichia coli, Bacillus subtilis, Salmonella typhimurium, or any bacterial strain capable of expressing heterologous peptides. If the peptide is made in yeast or bacteria, it may be necessary to modify the peptide produced therein, for example by phosphorylation or glycosylation of the appropriate sites, in order to obtain the functional peptide. Such covalent attachments may be accomplished using known chemical or enzymatic methods. The peptide may also be produced by operably linking an isolated polynucleotide of the disclosure to suitable control sequences in one or more insect expression vectors, and employing an insect expression system. Materials and methods for baculovirus/insect cell expression systems are commercially available in kit form from, e.g., Invitrogen, San Diego, Calif., U.S.A. (the MaxBac® kit), and such methods are well known in the art, as described in Summers and Smith, Texas Agricultural Experiment Station Bulletin No. 1555 (1987), and Luckow and Summers, Bio/Technology 6:47 (1988), incorporated herein by reference. As used herein, an insect cell capable of expressing a polynucleotide of the disclosure is “transformed.” Cell-free translation systems could also be employed to produce peptides using RNAs derived from polynucleotide constructs disclosed herein. A host cell that comprises an isolated polynucleotide of the disclosure, typically operably linked to at least one expression control sequence, is a “recombinant host cell.”

A peptide may be prepared by culturing transformed host cells under culture conditions suitable to express the recombinant peptide. The resulting expressed peptide may then be purified from such culture (e.g., from culture medium or cell extracts) using known purification processes, such as gel filtration and ion exchange chromatography. The purification of a peptide may also include an affinity column containing agents which will bind to the peptide; one or more column steps over such affinity resins as concanavalin A-agarose, Heparin-toyopearl® or Cibacrom blue 3GA Sepharose®; one or more steps involving hydrophobic interaction chromatography using such resins as phenyl ether, butyl ether, or propyl ether; or immunoaffinity chromatography. Alternatively, a peptide of the disclosure may also be expressed in a form that will facilitate purification. For example, it may be expressed as a fusion peptide, such as those of maltose binding peptide (MBP), glutathione-S-transferase (GST) or thioredoxin (TRX). Kits for expression and purification of such fusion peptides are commercially available from New England BioLab (Beverly, Mass.), Pharmacia (Piscataway, N.J.) and InVitrogen, respectively. A peptide can also be tagged with an epitope and subsequently purified by using a specific antibody directed to such epitope. One such epitope (“Flag”) is commercially available from Kodak (New Haven, Conn.). Finally, one or more reverse-phase high performance liquid chromatography (RP-HPLC) steps employing hydrophobic RP-HPLC media, e.g., silica gel having pendant methyl or other aliphatic groups, can be employed to further purify the peptide. Some or all of the foregoing purification steps, in various combinations, can also be employed to provide a substantially homogeneous isolated recombinant peptide. A peptide thus purified is substantially free of other mammalian peptides and is defined in accordance with the disclosure as a “purified peptide”; such purified peptides of the disclosure include purified antibodies that bind to Presenilin peptides of the disclosure, fragments, variants, binding partner, and the like. A peptide of the disclosure may also be expressed as a product of transgenic animals, e.g., as a component of the milk of transgenic cows, goats, pigs, or sheep which are characterized by somatic or germ cells containing a polynucleotide encoding the peptide.

It is also possible to utilize an affinity column comprising a peptide-binding peptide of the disclosure, such as a monoclonal antibody generated against peptides of the disclosure, to affinity-purify expressed peptides. These peptides can be removed from an affinity column using conventional techniques, e.g., in a high salt elution buffer and then dialyzed into a lower salt buffer for use or by changing pH or other components depending on the affinity matrix utilized, or be competitively removed using the naturally occurring substrate of the affinity moiety, such as a peptide derived from the disclosure. In this aspect of the disclosure, peptide-binding peptides, such as the anti-peptide antibodies of the disclosure or other peptides that can interact with a peptide of the disclosure, can be bound to a solid phase support such as a column chromatography matrix or a similar substrate suitable for identifying, separating, or purifying cells that express peptides of the disclosure on their surface.

Adherence of peptide-binding peptides to a solid phase contacting surface can be accomplished by any number of techniques, for example, magnetic microspheres can be coated with these peptide-binding peptides and held in the incubation vessel through a magnetic field. Suspensions of cell mixtures are contacted with the solid phase that has such peptide-binding peptides thereon. Cells having peptides of the disclosure on their surface bind to the fixed peptide-binding peptide and unbound cells then are washed away. This affinity-binding method is useful for purifying, screening, or separating such peptide-expressing cells from solution. Methods of releasing positively selected cells from the solid phase are known in the art and encompass, for example, the use of enzymes. Such enzymes are preferably non-toxic and non-injurious to the cells and are directed to cleaving the cell-surface binding partner. Alternatively, mixtures of cells suspected of containing peptide-expressing cells of the disclosure can first be incubated with a biotinylated peptide-binding peptide of the disclosure. Incubation periods are typically at least one hour in duration to ensure sufficient binding to peptides of the disclosure. The resulting mixture then is passed through a column packed with avidin-coated beads, whereby the high affinity of biotin for avidin provides the binding of the peptide-binding cells to the beads. Use of avidin-coated beads is known in the art (see Berenson, et al., 1986, J. Cell. Biochem., IOD:239). Wash of unbound material and the release of the bound cells is performed using conventional methods.

The desired degree of purity depends on the intended use of a peptide. A relatively high degree of purity is desired when a peptide is to be administered in vivo, for example. In such a case, peptides are purified such that no peptide bands corresponding to other peptides are detectable upon analysis by SDS-polyacrylamide gel electrophoresis (SDS-PAGE). It will be recognized by one skilled in the pertinent field that multiple bands corresponding to the peptide can be visualized by SDS-PAGE, due to differential glycosylation, differential post-translational processing, and the like. In some embodiments, a peptide of the disclosure is purified to substantial homogeneity, as indicated by a single peptide band upon analysis by SDS-PAGE. The peptide band can be visualized by silver staining, Coomassie blue staining, or (if the peptide is radiolabeled) by autoradiography.

### 5.5 Method of Using the Peptides and Pharmaceutical Compositions

In one aspect, provided herein is a method of attenuating the binding of beta-Amyloid precursor protein (β-APP) with presenilin-1 (PS-1) and/or presenilin-2 (PS-2) in a cell comprising contacting a cell with the peptide provided herein. In some embodiments, the peptide provided herein attenuates (e.g., partially attenuates) the binding of beta-amyloid precursor protein (β-APP) with presenilin-1 (PS-1) and/or presenilin-2 (PS-2) in a cell by at least about 10%. In some embodiments, the peptide provided herein attenuates the binding of beta-amyloid precursor protein (β-APP) with presenilin-1 (PS-1) and/or presenilin-2 (PS-2) in a cell by at least about 20%. In some embodiments, the peptide provided herein attenuates the binding of beta-amyloid precursor protein (β-APP) with presenilin-1 (PS-1) and/or presenilin-2 (PS-2) in a cell by at least about 30%. In some embodiments, the peptide provided herein attenuates the binding of beta-amyloid precursor protein (β-APP) with presenilin-1 (PS-1) and/or presenilin-2 (PS-2) in a cell by at least about 40%. In some embodiments, the peptide provided herein attenuates the binding of beta-amyloid precursor protein (β-APP) with presenilin-1 (PS-1) and/or presenilin-2 (PS-2) in a cell by at least about 50%. In some embodiments, the peptide provided herein attenuates the binding of beta-amyloid precursor protein (β-APP) with presenilin-1 (PS-1) and/or presenilin-2 (PS-2) in a cell by at least about 60%. In some embodiments, the peptide provided herein attenuates the binding of beta-amyloid precursor protein (β-APP) with presenilin-1 (PS-1) and/or presenilin-2 (PS-2) in a cell by at least about 70%. In some embodiments, the peptide provided herein attenuates the binding of beta-amyloid precursor protein (β-APP) with presenilin-1 (PS-1) and/or presenilin-2 (PS-2) in a cell by at least about 80%. In some embodiments, the peptide provided herein attenuates the binding of beta-amyloid precursor protein (β-APP) with presenilin-1 (PS-1) and/or presenilin-2 (PS-2) in a cell by at least about 90%. In some embodiments, the peptide provided herein attenuates the binding of beta-amyloid precursor protein (β-APP) with presenilin-1 (PS-1) and/or presenilin-2 (PS-2) in a cell by at least about 95%.

In one aspect, provided herein is a method of attenuating the production of amyloid β (Aβ) in a cell comprising contacting the cell with a peptide provided herein. In some embodiments, the peptide provided herein attenuates the production of Aβ in a cell by at least about 10%. In some embodiments, the peptide provided herein attenuates the production of Aβ in a cell by at least about 20%. In some embodiments, the peptide provided herein attenuates the production of Aβ in a cell by at least about 30%. In some embodiments, the peptide provided herein attenuates the production of Aβ in a cell by at least about 40%. In some embodiments, the peptide provided herein attenuates the production of Aβ in a cell by at least about 50%. In some embodiments, the peptide provided herein attenuates the production of Aβ in a cell by at least about 60%. In some embodiments, the peptide provided herein attenuates the production of Aβ in a cell by at least about 70%. In some embodiments, the peptide provided herein attenuates the production of Aβ in a cell by at least about 80%. In some embodiments, the peptide provided herein attenuates the production of Aβ in a cell by at least about 90%. In some embodiments, the peptide provided herein attenuates the production of Aβ in a cell by at least about 95%. In a specific embodiment, the cell is a retina cell.

In another aspect, provided herein is a method of attenuating the production of amyloid β (Aβ) in a subject comprising administering to the subject an effective amount of the peptide provided herein or a pharmaceutical composition comprising the same. In some embodiments, the peptide provided herein attenuates the production of Aβ in a subject's plasma by at least about 10%. In some embodiments, the peptide provided herein attenuates the production of Aβ in a subject's plasma by at least about 20%. In some embodiments, the peptide provided herein attenuates the production of Aβ in a subject's plasma by at least about 30%. In some embodiments, the peptide provided herein attenuates the production of Aβ in a subject's plasma by at least about 40%. In some embodiments, the peptide provided herein attenuates the production of Aβ in a subject's plasma by at least about 50%. In some embodiments, the peptide provided herein attenuates the production of Aβ in a subject's plasma by at least about 60%. In some embodiments, the peptide provided herein attenuates the production of Aβ in a subject's plasma by at least about 70%. In some embodiments, the peptide provided herein attenuates the production of Aβ in a subject's plasma by at least about 80%. In some embodiments, the peptide provided herein attenuates the production of Aβ in a subject's plasma by at least about 90%. In some embodiments, the peptide provided herein attenuates the production of Aβ in a subject's plasma by at least about 95%.

In some embodiments, the peptide provided herein attenuates the production of Aβ in a subject's CSF by at least about 10%. In some embodiments, the peptide provided herein attenuates the production of Aβ in a subject's CSF by at least about 20%. In some embodiments, the peptide provided herein attenuates the production of Aβ in a subject's CSF by at least about 30%. In some embodiments, the peptide provided herein attenuates the production of Aβ in a subject's CSF by at least about 40%. In some embodiments, the peptide provided herein attenuates the production of Aβ in a subject's CSF by at least about 50%. In some embodiments, the peptide provided herein attenuates the production of Aβ in a subject's CSF by at least about 60%. In some embodiments, the peptide provided herein attenuates the production of Aβ in a subject's CSF by at least about 70%. In some embodiments, the peptide provided herein attenuates the production of Aβ in a subject's CSF by at least about 80%. In some embodiments, the peptide provided herein attenuates the production of Aβ in a subject's CSF by at least about 90%. In some embodiments, the peptide provided herein attenuates the production of Aβ in a subject's CSF by at least about 95%.

In yet another aspect, provided herein is a method of attenuating an amyloid β activity in a cell comprising contacting the cell with the peptide provided herein. In yet another aspect, provided herein is a method of attenuating amyloid β activity in a subject comprising administering to the subject an effective amount of the peptide provided herein or a pharmaceutical composition comprising the same. A non-limiting example of an amyloid activity is amyloid β-induced or -mediated signaling. Thus, in certain embodiments, the peptides provided herein attenuates (e.g., partially attenuates) amyloid β-induced signaling. Another non-limiting example of amyloid β-induced signaling is interacting with (including blocking) receptors including but not limited to glucose transporters, NMDAR, AMPAR and acetylcholine receptors, activation of inflammatory signaling pathways, and the activation of one or more kinases including but not limited to GSK-3, CDK5, PKC, PKA and Erk1/2. Activities can include blocking ion channels, disruption of calcium homeostasis, mitochondrial oxidative stress, impaired energy metabolism, abnormal glucose regulation and/or neuronal cell death.

In some embodiments, the peptides provided herein attenuate an amyloid β activity (e.g., amyloid β induced signaling) in a cell by at least about 10%. In some embodiments, the peptides provided herein attenuate an amyloid β activity (e.g., amyloid β induced signaling) in a cell by at least about 20%. In some embodiments, the peptides provided herein attenuate an amyloid β activity (e.g., amyloid β induced signaling) in a cell by at least about 30%. In some embodiments, the peptides provided herein attenuate an amyloid β activity (e.g., amyloid β induced signaling) in a cell at least about 40%. In some embodiments, the peptides provided herein attenuate an amyloid β activity (e.g., amyloid β induced signaling) in a cell by at least about 50%. In some embodiments, the peptides provided herein attenuate an amyloid β activity (e.g., amyloid β induced signaling) in a cell by at least about 60%. In some embodiments, the peptides provided herein attenuate an amyloid β activity (e.g., amyloid β induced signaling) in a cell by at least about 70%. In some embodiments, the peptides provided herein attenuate an amyloid β activity (e.g., amyloid β induced signaling) in a cell by at least about 80%. In some embodiments, the peptides provided herein attenuate an amyloid β activity (e.g., amyloid β induced signaling) in a cell by at least about 90%. In some embodiments, the peptides provided herein attenuate an amyloid β activity (e.g., amyloid β induced signaling) in a cell by at least about 95%. In a specific embodiment, the cell is a retina cell.

In some embodiments, the peptides provided herein attenuate an amyloid β activity (e.g., amyloid β induced signaling) in a subject by at least about 10%. In some embodiments, the peptides provided herein attenuate an amyloid β activity (e.g., amyloid β induced signaling) in a subject by at least about 20%. In some embodiments, the peptides provided herein attenuate an amyloid β activity (e.g., amyloid β induced signaling) in a subject by at least about 30%. In some embodiments, the peptides provided herein attenuate an amyloid β activity (e.g., amyloid β induced signaling) in a subject at least about 40%. In some embodiments, the peptides provided herein attenuate an amyloid β activity (e.g., amyloid β induced signaling) in a subject by at least about 50%. In some embodiments, the peptides provided herein attenuate an amyloid β activity (e.g., amyloid β induced signaling) in a subject by at least about 60%. In some embodiments, the peptides provided herein attenuate an amyloid β activity (e.g., amyloid β induced signaling) in a subject by at least about 70%. In some embodiments, the peptides provided herein attenuate an amyloid β activity (e.g., amyloid β induced signaling) in a subject by at least about 80%. In some embodiments, the peptides provided herein attenuate an amyloid β activity (e.g., amyloid β induced signaling) in a subject by at least about 90%. In some embodiments, the peptides provided herein attenuate an amyloid β activity (e.g., amyloid β induced signaling) in a subject by at least about 95%.

In one embodiment, the amyloid β is amyloid β 36, amyloid β 37, amyloid β 38, amyloid β 39, amyloid β 40, amyloid β 41, amyloid β 42, amyloid β 43, amyloid β 44, amyloid β 45, amyloid β 46, amyloid β 47, amyloid β 48, amyloid β 49, amyloid β 50, amyloid β 51, or amyloid β 52, or a combination thereof. In another embodiment, the amyloid β is amyloid β 40. In yet another embodiment, the amyloid β is amyloid β 42.

In yet another aspect, provided herein is a method of attenuating the production of Tau protein is a cell comprising contacting the cell with the peptide provided herein. In some embodiments, the peptide provided herein attenuates the production of Tau in a cell by at least about 10%. In some embodiments, the peptide provided herein attenuates the production of Tau in a cell by at least about 20%. In some embodiments, the peptide provided herein attenuates the production of Tau in a cell by at least about 30%. In some embodiments, the peptide provided herein attenuates the production of Tau in a cell by at least about 40%. In some embodiments, the peptide provided herein attenuates the production of Tau in a cell by at least about 50%. In some embodiments, the peptide provided herein attenuates the production of Tau in a cell by at least about 60%. In some embodiments, the peptide provided herein attenuates the production of Tau in a cell by at least about 70%. In some embodiments, the peptide provided herein attenuates the production of Tau in a cell by at least about 80%. In some embodiments, the peptide provided herein attenuates the production of Tau in a cell by at least about 90%. In some embodiments, the peptide provided herein attenuates the production of Tau in a cell by at least about 95%. In a specific embodiment, the cell is a retina cell.

In yet another aspect, provided herein is a method of attenuating the production of tau protein in a subject comprising administering to the subject the peptide provided herein or a pharmaceutical composition comprising the same. In some embodiments, the peptide provided herein attenuates the amount of Tau in a subject's plasma by at least about 10%. In some embodiments, the peptide provided herein attenuates the amount of Tau in a subject's plasma by at least about 20%. In some embodiments, the peptide provided herein attenuates the amount of Tau in a subject's plasma by at least about 30%. In some embodiments, the peptide provided herein attenuates the amount of Tau in a subject's plasma by at least about 40%. In some embodiments, the peptide provided herein attenuates the amount of Tau in a subject's plasma by at least about 50%. In some embodiments, the peptide provided herein attenuates the amount of Tau in a subject's plasma by at least about 60%. In some embodiments, the peptide provided herein attenuates the amount of Tau in a subject's plasma by at least about 70%. In some embodiments, the peptide provided herein attenuates the amount of Tau in a subject's plasma by at least about 80%. In some embodiments, the peptide provided herein attenuates the amount of Tau in a subject's plasma by at least about 90%. In some embodiments, the peptide provided herein attenuates the amount of Tau in a subject's plasma by at least about 95%.

In yet another aspect, provided herein is a method of attenuating a tau protein activity in a cell comprising contacting the cell with the peptide provided herein. In yet another aspect, provided herein is a method of attenuating a tau protein activity in a subject comprising administering to the subject an effective amount of the peptide provided herein or a pharmaceutical composition comprising the same. A non-limiting example of a tau protein activity is a tau protein-induced or -mediated signaling. Thus, in certain embodiments, the peptides provided herein attenuates (e.g., partially attenuates) tau protein-induced signaling. Non-limiting examples of a tau protein activity include interacting with tubulin to stabilize microtubules, formation of helical and/or straight filaments, activation of inflammatory signaling pathways and impaired insulin signaling in the brain.

In some embodiments, the peptide provided herein attenuates a tau protein activity (e.g., tau protein-induced signaling) in a cell by at least about 10%. In some embodiments, the peptide provided herein attenuates a tau protein activity (e.g., tau protein-induced signaling) in a cell by at least about 20%. In some embodiments, the peptide provided herein attenuates a tau protein activity (e.g., tau protein-induced signaling) in a cell by at least about 30%. In some embodiments, the peptide provided herein attenuates a tau protein activity (e.g., tau protein-induced signaling) in a cell at least about 40%. In some embodiments, the peptide provided herein attenuates a tau protein activity (e.g., tau protein-induced signaling) in a cell by at least about 50%. In some embodiments, the peptide provided herein attenuates a tau protein activity (e.g., tau protein-induced signaling) in a cell by at least about 60%. In some embodiments, the peptide provided herein attenuates a tau protein activity (e.g., tau protein-induced signaling) in a cell by at least about 70%. In some embodiments, the peptide provided herein attenuates a tau protein activity (e.g., tau protein-induced signaling) in a cell by at least about 80%. In some embodiments, the peptide provided herein attenuates a tau protein activity (e.g., tau protein-induced signaling) in a cell by at least about 90%. In some embodiments, the peptide provided herein attenuates a tau protein activity (e.g., tau protein-induced signaling) in a cell by at least about 95%. In a specific embodiment, the cell is a retina cell.

In some embodiments, the peptide provided herein attenuates a tau protein activity (e.g., tau protein-induced signaling) in a subject by at least about 10%. In some embodiments, the peptide provided herein attenuates a tau protein activity (e.g., tau protein-induced signaling) in a subject by at least about 20%. In some embodiments, the peptide provided herein attenuates a tau protein activity (e.g., tau protein-induced signaling) in a subject by at least about 30%. In some embodiments, the peptide provided herein attenuates a tau protein activity (e.g., tau protein-induced signaling) in a subject at least about 40%. In some embodiments, the peptide provided herein attenuates a tau protein activity (e.g., tau protein-induced signaling) in a subject by at least about 50%. In some embodiments, the peptide provided herein attenuates a tau protein activity (e.g., tau protein-induced signaling) in a subject by at least about 60%. In some embodiments, the peptide provided herein attenuates a tau protein activity (e.g., tau protein-induced signaling) in a subject by at least about 70%. In some embodiments, the peptide provided herein attenuates a tau protein activity (e.g., tau protein-induced signaling) in a subject by at least about 80%. In some embodiments, the peptide provided herein attenuates a tau protein activity (e.g., tau protein-induced signaling) in a subject by at least about 90%. In some embodiments, the peptide provided herein attenuates a tau protein activity (e.g., tau protein-induced signaling) in a subject by at least about 95%.

In yet another aspect, provided herein is a method for treating or preventing a disease or disorder in a subject, comprising administering to the subject a therapeutically effective amount of a peptide provided herein or a pharmaceutical composition comprising the same. As used herein, a disease or disorder also includes one or more symptoms of the disease or disorder.

In certain embodiments, the disease or disorder is an amyloid (or amyloid β) related disease or disorder. In certain embodiments, the disease or disorder is a disease or disorder associated with amyloid fibril formation, aggregation or deposition. In certain embodiments, the disease or disorder is a neurological disease. In certain embodiments, the disease or disorder is a neurodegenerative disease.

In some embodiments, the disease or disorder is an ocular disorder. In some embodiments, the ocular disorder is related to accumulation of amyloid β. In some embodiments, the ocular disorder is macular degeneration. In some embodiments, the ocular disorder is age-related macular degeneration.

In some embodiments, the peptides of the disclosure may be administered therapeutically or prophylactically to treat diseases associated with amyloid fibril formation, aggregation or deposition, regardless of the clinical setting. The peptides of the disclosure may act to modulate the course of an amyloid β related disease using any of the following mechanisms, such as, for example but not limited to, slowing the rate of amyloid fibril formation or deposition; lessening the degree of amyloid deposition; inhibiting, reducing, or preventing amyloid fibril formation; inhibiting amyloid induced inflammation; enhancing the clearance of amyloid from, for example, the brain; or protecting cells from amyloid induced (oligomers or fibrillar) toxicity. Modulation of amyloid deposition is intended to encompass prevention or stopping of amyloid formation or accumulation, inhibition or slowing down of further amyloid aggregation in a subject with ongoing amyloidosis, e.g., already having amyloid aggregates, and reducing or reversing of amyloid aggregates in a subject with ongoing amyloidosis. Modulation of amyloid aggregation is determined relative to an untreated subject or relative to the treated subject prior to treatment, e.g., determined by clinically measurable improvement, or in the case of a subject with brain amyloidosis, e.g., an Alzheimer's or cerebral amyloid angiopathy subject, stabilization of cognitive function or prevention of a further decrease in cognitive function (i.e., preventing, slowing, or stopping disease progression), or improvement of parameters such as the concentration of Aβ or tau in the CSF.

While Alzheimer's disease of the familial or the sporadic type is the major dementia found in the aging population, other types of dementia are also found. These include but are not limited to: the fronto-temporal degeneration associated with Pick's disease, vascular dementia, senile dementia of Lewy body type, dementia of Parkinsonism with frontal atrophy, progressive supranuclear palsy and corticobasal degeneration and Down syndrome associated Alzheimers' disease. Plaque formation is also seen in the spongiform encephalopathies such as CJD, scrapie and BSE. The disclosure is directed to treatment of such neurodegenerative diseases, particularly those involving neurotoxic protein plaques, e.g., amyloid plaques.

Down syndrome is a serious human disorder that occurs with an incidence of 1 in 800 live births. It is associated with the presence in affected individuals of an extra copy of chromosome 21 (trisomy 21). The β-amyloid precursor protein (β-APP) gene is encoded on chromosome 21, very close to the Down syndrome locus. All patients with Down syndrome, if they survive beyond 40 years, develop Alzheimer's-like dementia and the deposition of Aβ in their brains. There is good reason, therefore, to propose that the over-production of Aβ is connected directly with the occurrence of the dementia in both AD and Down syndrome. Therefore, the nature of the identification of therapeutic agents for the amelioration of the symptoms of AD will also be useful for the amelioration of the symptoms of Down syndrome.

“Dementia” refers to a general mental deterioration due to organic or psychological factors; characterized by disorientation, impaired memory, judgment, and intellect, and a shallow labile affect. Dementia herein includes vascular dementia, ischemic vascular dementia (IVD), frontotemporal dementia (FTD), Lewy body dementia, Alzheimer's dementia, etc. The most common form of dementia among older people is Alzheimer's disease (AD).

The peptide provided herein may be used to treat mild cognitive impairment. Mild Cognitive Impairment (“MCI”) is a condition characterized by a state of mild but measurable impairment in thinking skills, which is not necessarily associated with the presence of dementia. MCI frequently, but not necessarily, precedes Alzheimer's disease.

Additionally, abnormal accumulation of APP and of amyloid-β protein in muscle fibers has been implicated in the pathology of sporadic inclusion body myositis (IBM) (see Askanas et al., 1996, Proc. Natl. Acad. Sci. USA 93: 1314-1319; Askanas, V. et al., 1995, Current Opinion in Rheumatology 7: 486-496). Accordingly, the peptide provided herein may be used prophylactically or therapeutically in the treatment of disorders in which amyloid-β protein is abnormally deposited at non-neurological locations, such as treatment of EBM by delivery of the peptide to muscle fibers.

Additionally, it has been shown that Aβ is associated with abnormal extracellular deposits, known as drusen, that accumulate along the basal surface of the retinal pigmented epithelium in individuals with age-related macular degeneration (ARMD). ARMD is a cause of irreversible vision loss in older individuals. It is believed that Aβ deposition could be an important component of the local inflammatory events that contribute to atrophy of the retinal pigmented epithelium, drusen biogenesis, and the pathogenesis of ARMD (Johnson, et al., 2002, Proc. Natl. Acad. Sci. USA 99(18), 11830-5).

Accordingly, the disclosure relates generally to methods of treating or preventing an amyloid-related disease or disorder in a subject (preferably a human) comprising administering to the subject a therapeutic amount of a peptide provided herein, such that amyloid fibril formation or deposition, neurodegeneration, or cellular toxicity is reduced or inhibited. In another embodiment, the disclosure relates to a method of treating or preventing an amyloid-related disease in a subject (preferably a human) comprising administering to the subject a therapeutic amount of a peptide provided herein, such that cognitive function is improved or stabilized or further deterioration in cognitive function is prevented, slowed, or stopped in patients with brain amyloidosis, e.g., Alzheimer's disease, Down syndrome or cerebral amyloid angiopathy.

In certain embodiments, the disease or disorder is selected from a group consisting of Parkinson's disease (PD), Alzheimer's disease (AD), traumatic brain injury (TBI), amyotrophic lateral sclerosis (ALS), multiple sclerosis (MS), or dementia. In certain embodiments, the disease or disorder is Parkinson's disease. In certain embodiments, the disease or disorder is traumatic brain injury. In certain embodiments, the disease or disorder is amyotrophic lateral sclerosis. In certain embodiments, the disease or disorder is multiple sclerosis. In certain embodiments, the disease or disorder is dementia.

In certain embodiments, the disease or disorder is dementia or dementia related disease or disorder selected from a group consisting of frontotemporal dementia, fronto-temporal degeneration associated with Pick's disease, vascular dementia, corticobasal degeneration, ischemic vascular dementia (IVD), Lewy body dementia, and Alzheimer's dementia.

In certain embodiments, the disease or disorder is an ocular disorder. In certain embodiments, the disorder, disease, or condition is Down syndrome.

In certain embodiments, the disease or disorder is selected from a group consisting of transmissible spongiform encephalopathies (such as scrapie in sheep, Creutzfeldt-Jakob disease (CJD) in humans, bovine spongiform encephalopathy (BSE) in cattles), cerebral amyloid angiopathy, hereditary cerebral hemorrhage with amyloidosis, mild cognitive impairment, sporadic inclusion body myositis and age-related macular degeneration.

In certain embodiments, the disease or disorder is Alzheimer's disease. In certain embodiments, the Alzheimer's disease is Stage 1 AD (no impairment). In certain embodiments, the Alzheimer's disease is Stage 2 AD (very mild decline). In certain embodiments, the Alzheimer's disease is Stage 3 AD (mild decline). In certain embodiments, the Alzheimer's disease is Stage 4 AD (moderate decline). In certain embodiments, the Alzheimer's disease is Stage 5 AD (moderately severe decline). In certain embodiments, the Alzheimer's disease is Stage 6 AD (severe decline). In certain embodiments, the Alzheimer's disease is Stage 7 AD (very severe decline).

In certain embodiments, the disorder, disease, or condition is a disorder, disease, or condition mediated by a tau protein. In certain embodiments, the disorder, disease, or condition mediated by a tau protein is tauopathy. In certain embodiments, the disorder, disease, or condition mediated by a tau protein is Alzheimer's disease.

The methods provided herein encompass treating a subject regardless of patient's age, although some diseases or disorders are more common in certain age groups.

Depending on the disease to be treated and the subject's condition, a peptide provided herein or a pharmaceutical composition comprising same can be administered by oral, parenteral (e.g., intramuscular, intraperitoneal, intravenous, CIV, intracisternal injection or infusion, subcutaneous injection, or implant), inhalation, nasal, vaginal, rectal, sublingual, or topical (e.g., transdermal or local) routes of administration. A peptide provided herein or a pharmaceutical composition comprising same can be formulated, alone or together, in suitable dosage unit with pharmaceutically acceptable excipients, carriers, adjuvants and vehicles, appropriate for each route of administration. More description of the administration and dosing is provided in Section 5.6 below.

It will be understood, however, that the specific dose level and frequency of dosage for any particular subject can be varied and will depend upon a variety of factors including the activity of the specific peptide employed, the metabolic stability and length of action of that peptide, the age, body weight, general health, sex, diet, mode and time of administration, rate of excretion, drug combination, the severity of the particular condition, and the host undergoing therapy.

In certain embodiments, the subject is a mammal. In certain embodiments, the subject is a human.

A peptide provided herein or a pharmaceutical composition comprising same can also be combined or used in combination with other therapeutic agents useful in the treatment and/or prevention of a disorder, disease, or condition described herein.

As used herein, the term “in combination” includes the use of more than one therapy (e.g., one or more prophylactic and/or therapeutic agents). However, the use of the term “in combination” does not restrict the order in which therapies (e.g., prophylactic and/or therapeutic agents) are administered to a subject with a disease or disorder. A first therapy (e.g., a prophylactic or therapeutic agent such as a peptide provided herein) can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapy (e.g., a prophylactic or therapeutic agent) to the subject. Triple therapy is also contemplated herein.

In some embodiments, the route of administration of a peptide provided herein or a pharmaceutical composition comprising same is independent of the route of administration of a second therapy. In one embodiment, a peptide provided herein or a pharmaceutical composition comprising same is administered orally. In another embodiment, a peptide provided herein or a pharmaceutical composition comprising same is administered intravenously. Thus, in accordance with these embodiments, a peptide provided herein or a pharmaceutical composition comprising same is administered orally or intravenously, and the second therapy can be administered orally, parenterally, intraperitoneally, intravenously, intraarterially, transdermally, sublingually, intramuscularly, rectally, transbuccally, intranasally, liposomally, via inhalation, vaginally, intraoccularly, via local delivery by catheter or stent, subcutaneously, intraadiposally, intraarticularly, intrathecally, or in a slow release dosage form. In one embodiment, a peptide provided herein or a pharmaceutical composition comprising same, and a second therapy are administered by the same mode of administration, orally or by IV. In another embodiment, a peptide provided herein or a pharmaceutical composition comprising same is administered by one mode of administration, e.g., by IV, whereas the second agent is administered by another mode of administration, e.g., orally.

In certain embodiments, each method provided herein may independently, further comprise the step of administering a second therapeutic agent.

### 5.6 Methods of Administration and Dosing

In a specific embodiment, provided herein is a composition for use in the prevention and/or treatment of a disease or condition comprising a peptide provided herein. In one embodiment, provided herein is a composition for use in the prevention of a disease or condition, wherein the composition comprises a peptide provided herein. In one embodiment, provided herein is a composition for use in the treatment of a disease or condition, wherein the composition comprises a peptide provided herein. In certain embodiments, the disease or disorder is an amyloid (or amyloid β) related disease or disorder. In certain embodiments, the disease or disorder is a disease or disorder associated with amyloid fibril formation, aggregation or deposition. In certain embodiments, the disease or disorder is a neurological disease. In certain embodiments, the disease or disorder is a neurodegenerative disease. In certain embodiments, the disease or disorder is selected from a group consisting of Parkinson's disease (PD), Alzheimer's disease (AD), traumatic brain injury (TBI), amyotrophic lateral sclerosis (ALS), multiple sclerosis (MS), or dementia. In certain embodiments, the disease or disorder is dementia or dementia related disease or disorder selected from a group consisting of frontotemporal dementia, fronto-temporal degeneration associated with Pick's disease, vascular dementia, corticobasal degeneration, ischemic vascular dementia (IVD), Lewy body dementia, and Alzheimer's dementia. In certain embodiments, the disease or disorder is an ocular disorder. In certain embodiments, the ocular disorder is macular degeneration. In certain embodiments, the ocular disorder is age-related macular degeneration. In certain embodiments, the disorder, disease, or condition is Down syndrome. In certain embodiments, the disease or disorder is selected from a group consisting of transmissible spongiform encephalopathies (such as scrapie in sheep, Creutzfeldt-Jakob disease (CJD) in humans, bovine spongiform encephalopathy (BSE) in cattles), cerebral amyloid angiopathy, hereditary cerebral hemorrhage with amyloidosis, mild cognitive impairment, sporadic inclusion body myositis and age-related macular degeneration.

In certain embodiments, the subject is a subject in need thereof. In some embodiments, the subject has the disease or condition. In other embodiments, the subject is at risk of having the disease or condition. In some embodiments, the administration results in the prevention, management, treatment or amelioration of the disease or condition.

In one embodiment, provided herein is a composition for use in the prevention and/or treatment of a symptom of a disease or condition, wherein the composition comprises a peptide provided herein. In one embodiment, provided herein is a composition for use in the prevention of a symptom of a disease or condition, wherein the composition comprises a peptide provided herein. In one embodiment, provided herein is a composition for use in the treatment of a symptom of a disease or condition, wherein the composition comprises a peptide provided herein. In certain embodiments, the disease or disorder is an amyloid (or amyloid β) related disease or disorder. In certain embodiments, the disease or disorder is a disease or disorder associated with amyloid fibril formation, aggregation or deposition. In certain embodiments, the disease or disorder is a neurological disease. In certain embodiments, the disease or disorder is a neurodegenerative disease. In certain embodiments, the disease or disorder is selected from a group consisting of Parkinson's disease (PD), Alzheimer's disease (AD), traumatic brain injury (TBI), amyotrophic lateral sclerosis (ALS), multiple sclerosis (MS), or dementia. In certain embodiments, the disease or disorder is dementia or dementia related disease or disorder selected from a group consisting of frontotemporal dementia, fronto-temporal degeneration associated with Pick's disease, vascular dementia, corticobasal degeneration, ischemic vascular dementia (IVD), Lewy body dementia, and Alzheimer's dementia. In certain embodiments, the disease or disorder is an ocular disorder. In certain embodiments, the ocular disorder is macular degeneration. In certain embodiments, the ocular disorder is age-related macular degeneration. In certain embodiments, the disorder, disease, or condition is Downs syndrome. In certain embodiments, the disease or disorder is selected from a group consisting of transmissible spongiform encephalopathies (such as scrapie in sheep, Creutzfeldt-Jakob disease (CJD) in humans, bovine spongiform encephalopathy (BSE) in cattles), cerebral amyloid angiopathy, hereditary cerebral hemorrhage with amyloidosis, mild cognitive impairment, sporadic inclusion body myositis and age-related macular degeneration.

In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

In another embodiment, provided herein is a method of preventing and/or treating a disease or condition in a subject, comprising administering an effective amount of a peptide provided herein. In one embodiment, provided herein is a method of preventing a disease or condition in a subject, comprising administering an effective amount of a peptide provided herein. In one embodiment, provided herein is a method of treating a disease or condition in a subject, comprising administering an effective amount of a peptide provided herein. In certain embodiments, the disease or disorder is an amyloid (or amyloid β) related disease or disorder. In certain embodiments, the disease or disorder is a disease or disorder associated with amyloid fibril formation, aggregation or deposition. In certain embodiments, the disease or disorder is a neurological disease. In certain embodiments, the disease or disorder is a neurodegenerative disease. In certain embodiments, the disease or disorder is selected from a group consisting of Parkinson's disease (PD), Alzheimer's disease (AD), traumatic brain injury (TBI), amyotrophic lateral sclerosis (ALS), multiple sclerosis (MS), or dementia. In certain embodiments, the disease or disorder is dementia or dementia related disease or disorder selected from a group consisting of frontotemporal dementia, fronto-temporal degeneration associated with Pick's disease, vascular dementia, corticobasal degeneration, ischemic vascular dementia (IVD), Lewy body dementia, and Alzheimer's dementia. In certain embodiments, the disease or disorder is an ocular disorder. In certain embodiments, the ocular disorder is macular degeneration. In certain embodiments, the ocular disorder is age-related macular degeneration. In certain embodiments, the disorder, disease, or condition is Downs syndrome. In certain embodiments, the disease or disorder is selected from a group consisting of transmissible spongiform encephalopathies (such as scrapie in sheep, Creutzfeldt-Jakob disease (CJD) in humans, bovine spongiform encephalopathy (BSE) in cattles), cerebral amyloid angiopathy, hereditary cerebral hemorrhage with amyloidosis, mild cognitive impairment, sporadic inclusion body myositis and age-related macular degeneration.

In certain embodiments, the subject is a subject in need thereof. In some embodiments, the subject has the disease or condition. In other embodiments, the subject is at risk of having the disease or condition. In some embodiments, the administration results in the prevention or treatment of the disease or condition.

In another embodiment, provided herein is a method of preventing and/or treating a symptom of a disease or condition in a subject, comprising administering an effective amount of a peptide provided herein. In one embodiment, provided herein is a method of preventing a symptom of a disease or condition in a subject, comprising administering an effective amount of a peptide provided herein. In one embodiment, provided herein is a method of treating a symptom of a disease or condition in a subject, comprising administering an effective amount of a peptide provided herein. In certain embodiments, the disease or disorder is an amyloid (or amyloid β) related disease or disorder. In certain embodiments, the disease or disorder is a disease or disorder associated with amyloid fibril formation, aggregation or deposition. In certain embodiments, the disease or disorder is a neurological disease. In certain embodiments, the disease or disorder is a neurodegenerative disease. In certain embodiments, the disease or disorder is selected from a group consisting of Parkinson's disease (PD), Alzheimer's disease (AD), traumatic brain injury (TBI), amyotrophic lateral sclerosis (ALS), multiple sclerosis (MS), or dementia. In certain embodiments, the disease or disorder is dementia or dementia related disease or disorder selected from a group consisting of frontotemporal dementia, fronto-temporal degeneration associated with Pick's disease, vascular dementia, corticobasal degeneration, ischemic vascular dementia (IVD), Lewy body dementia, and Alzheimer's dementia. In certain embodiments, the disease or disorder is an ocular disorder. In certain embodiments, the ocular disorder is macular degeneration. In certain embodiments, the ocular disorder is age-related macular degeneration. In certain embodiments, the disorder, disease, or condition is Downs syndrome. In certain embodiments, the disease or disorder is selected from a group consisting of transmissible spongiform encephalopathies (such as scrapie in sheep, Creutzfeldt-Jakob disease (CJD) in humans, bovine spongiform encephalopathy (BSE) in cattles), cerebral amyloid angiopathy, hereditary cerebral hemorrhage with amyloidosis, mild cognitive impairment, sporadic inclusion body myositis and age-related macular degeneration.

In certain embodiments, the subject is a subject in need thereof. In some embodiments, the subject has the disease or condition. In other embodiments, the subject is at risk of having the disease or condition. In some embodiments, the administration results in the prevention or treatment of the symptom of the disease or condition.

Also provided herein are methods of preventing and/or treating a disease or condition by administrating to a subject of an effective amount of a peptide provided herein, or pharmaceutical composition comprising a peptide provided herein. In one aspect, the peptide is substantially purified (i.e., substantially free from substances that limit its effect or produce undesired side-effects). The subject administered a therapy can be a mammal such as non-primate (e.g., cows, pigs, horses, cats, dogs, rats etc.) or a primate (e.g., a monkey, such as a cynomolgus monkey, or a human). In a one embodiment, the subject is a human. In another embodiment, the subject is a human with a disease or condition.

Various delivery systems are known and can be used to administer a prophylactic or therapeutic agent (e.g., a peptide provided herein), including, but not limited to, encapsulation in liposomes, microparticles, microcapsules, recombinant cells capable of expressing the peptide, receptor-mediated endocytosis (see Wu and Wu, 1987, J. Biol. Chem. 262:4429-4432), construction of a nucleic acid as part of a retroviral or other vector, etc. Methods of administering a prophylactic or therapeutic agent (e.g., a peptide provided herein), or pharmaceutical composition include, but are not limited to, parenteral administration (e.g., intradermal, intramuscular, intraperitoneal, intravenous and subcutaneous), epidural, and mucosal (e.g., intranasal and oral routes). In a specific embodiment, a prophylactic or therapeutic agent (e.g., a peptide provided herein), or a pharmaceutical composition is administered intranasally, intramuscularly, intravenously, or subcutaneously. The prophylactic or therapeutic agents, or compositions may be administered by any convenient route, for example by infusion or bolus injection, by absorption through epithelial or mucocutaneous linings (e.g., oral mucosa, intranasal mucosa, rectal and intestinal mucosa, etc.) and may be administered together with other biologically active agents. Administration can be systemic or local. In addition, pulmonary administration can also be employed, e.g., by use of an inhaler or nebulizer, and formulation with an aerosolizing agent. See, e.g., U.S. Pat. Nos. 6,019,968, 5,985,320, 5,985,309, 5,934,272, 5,874,064, 5,855,913, 5,290,540, and 4,880,078; and PCT Publication Nos. WO 92/19244, WO 97/32572, WO 97/44013, WO 98/31346, and WO 99/66903, each of which is incorporated herein by reference their entirety.

In a specific embodiment, it may be desirable to administer a prophylactic or therapeutic agent, or a pharmaceutical composition provided herein locally to the area in need of treatment. This may be achieved by, for example, and not by way of limitation, local infusion, by topical administration (e.g., by intranasal spray), by injection, or by means of an implant, said implant being of a porous, non-porous, or gelatinous material, including membranes, such as sialastic membranes, or fibers. In some embodiments, when administering a peptide provided herein, care must be taken to use materials to which the peptide does not absorb.

In another embodiment, a prophylactic or therapeutic agent, or a composition provided herein can be delivered in a vesicle, in particular a liposome (see Langer, 1990, Science 249:1527-1533; Treat et al., 1989, in Liposomes in the Therapy of Infectious Disease and Cancer, pp. 353-365; Lopez-Berestein, ibid., pp. 317-327; see generally ibid).

In another embodiment, a prophylactic or therapeutic agent, or a composition provided herein can be delivered in a controlled release or sustained release system. In one embodiment, a pump may be used to achieve controlled or sustained release (see Langer, supra; Sefton, 1987, CRC Crit. Ref. Biomed. Eng. 14:20; Buchwald et al., 1980, Surgery 88:507; Saudek et al., 1989, N. Engl. J. Med. 321:574). In another embodiment, polymeric materials can be used to achieve controlled or sustained release of a prophylactic or therapeutic agent (e.g., a peptide provided herein) or a composition provided herein (see Langer and Wise eds., 1974, Medical Applications of Controlled Release; Smolen and Ball eds., 1984, Controlled Drug Bioavailability, Drug Product Design and Performance; Ranger and Peppas, 1983, J., Macromol. Sci. Rev. Macromol. Chem. 23:61; Levy et al., 1985, Science 228:190; During et al., 1989, Ann. Neurol. 25:351; Howard et al., 1989, J. Neurosurg. 7 1:105; U.S. Pat. Nos. 5,679,377; 5,916,597; 5,912,015; 5,989,463; 5,128,326; PCT Publication No. WO 99/15154; and PCT Publication No. WO 99/20253) Examples of polymers used in sustained release formulations include, but are not limited to, poly(2-hydroxy ethyl methacrylate), poly(methyl methacrylate), poly(acrylic acid), poly(ethylene-co-vinyl acetate), poly(methacrylic acid), polyglycolides (PLG), polyanhydrides, poly(N-vinyl pyrrolidone), poly(vinyl alcohol), polyacrylamide, poly(ethylene glycol), polylactides (PLA), poly(lactide-co-glycolides) (PLGA), and polyorthoesters. In an embodiment, the polymer used in a sustained release formulation is inert, free of leachable impurities, stable on storage, sterile, and biodegradable. In yet another embodiment, a controlled or sustained release system can be placed in proximity of the therapeutic target, i.e., the nasal passages or lungs, thus requiring only a fraction of the systemic dose (see Goodson, in Medical Applications of Controlled Release, supra, vol. 2, pp. 115-138 (1984)). Controlled release systems are discussed in the review by Langer (1990, Science 249:1527-1533). Any technique known to one of skill in the art can be used to produce sustained release formulations comprising one or more peptides provided herein (See U.S. Pat. No. 4,526,938, PCT publication WO 91/05548, PCT publication WO 96/20698; Ning et al., 1996, Radiotherapy & Oncology 39:179-189; Song et al., 1995, PDA Journal of Pharmaceutical Science & Technology 50:372-397; Cleek et al., 1997, Pro. Int'l. Symp. Control. Rel. Bioact. Mater. 24:853-854, and Lam et al., 1997, Proc. Int'l. Symp. Control Rel. Bioact. Mater. 24:759-760, each of which is incorporated herein by reference in their entirety).

In a specific embodiment, where the composition provided herein is a nucleic acid encoding a prophylactic or therapeutic agent (e.g., a peptide provided herein or a parental peptide thereof), the nucleic acid can be administered in vivo to promote expression of its encoded prophylactic or therapeutic agent, by constructing it as part of an appropriate nucleic acid expression vector and administering it so that it becomes intracellular, e.g., by use of a retroviral vector (see U.S. Pat. No. 4,980,286), or by direct injection, or by use of microparticle bombardment (e.g., a gene gun; Biolistic, Dupont), or coating with lipids or cell surface receptors or transfecting agents, or by administering it in linkage to a homeobox-like peptide which is known to enter the nucleus (see Joliot et al., 1991, Proc. Natl. Acad. Sci. USA 88:1864-1868), etc. Alternatively, a nucleic acid can be introduced intracellularly and incorporated within host cell DNA for expression by homologous recombination.

In a specific embodiment, a composition provided herein comprises one, two or more peptides provided herein. In another embodiment, a composition provided herein comprises one, two or more peptides provided herein and a prophylactic or therapeutic agent other than peptides provided herein. In one embodiment, the agents are known to be useful for or have been or are currently used for the prevention, management, treatment and/or amelioration of a disease or condition. In addition to prophylactic or therapeutic agents, the compositions provided herein may also comprise an excipient.

The compositions provided herein include bulk drug compositions useful in the manufacture of pharmaceutical compositions (e.g., compositions that are suitable for administration to a subject or patient) that can be used in the preparation of unit dosage forms. In an embodiment, a composition provided herein is a pharmaceutical composition. Such compositions comprise a prophylactically or therapeutically effective amount of one or more prophylactic or therapeutic agents (e.g., peptides provided herein or other prophylactic or therapeutic agent), and a pharmaceutically acceptable excipient. The pharmaceutical compositions can be formulated to be suitable for the route of administration to a subject.

In a specific embodiment, the term “excipient” can also refer to a diluent, adjuvant (e.g., Freunds' adjuvant (complete or incomplete) or vehicle. Pharmaceutical excipients can be sterile liquids, such as water and oils, including those of petroleum, animal, vegetable or synthetic origin, such as peanut oil, soybean oil, mineral oil, sesame oil and the like. Water is an exemplary excipient when the pharmaceutical composition is administered intravenously. Saline solutions and aqueous dextrose and glycerol solutions can also be employed as liquid excipients, particularly for injectable solutions. Suitable pharmaceutical excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like. The composition, if desired, can also contain minor amounts of wetting or emulsifying agents, or pH buffering agents. These compositions can take the form of solutions, suspensions, emulsion, tablets, pills, capsules, powders, sustained-release formulations and the like. Oral formulation can include standard excipients such as pharmaceutical grades of mannitol, lactose, starch, magnesium stearate, sodium saccharine, cellulose, magnesium carbonate, etc. Examples of suitable pharmaceutical excipients are described in Remington's Pharmaceutical Sciences (1990) Mack Publishing Co., Easton, Pa. Such compositions will contain a prophylactically or therapeutically effective amount of the peptide provided herein, such as in purified form, together with a suitable amount of excipient so as to provide the form for proper administration to the patient. The formulation should suit the mode of administration.

In an embodiment, the composition is formulated in accordance with routine procedures as a pharmaceutical composition adapted for intravenous administration to human beings. Typically, compositions for intravenous administration are solutions in sterile isotonic aqueous buffer. Where necessary, the composition may also include a solubilizing agent and a local anesthetic such as lignocamne to ease pain at the site of the injection. Such compositions, however, may be administered by a route other than intravenous.

Generally, the ingredients of compositions provided herein are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients may be mixed prior to administration.

A peptide provided herein can be packaged in a hermetically sealed container such as an ampoule or sachette indicating the quantity of peptide. In one embodiment, the peptide is supplied as a dry sterilized lyophilized powder or water free concentrate in a hermetically sealed container and can be reconstituted, e.g., with water or saline to the appropriate concentration for administration to a subject. The lyophilized peptide can be stored at between 2 and 8° C. in its original container and the peptide can be administered within 12 hours, such as within 6 hours, within 5 hours, within 3 hours, or within 1 hour after being reconstituted. In an alternative embodiment, a peptide provided herein is supplied in liquid form in a hermetically sealed container indicating the quantity and concentration of the peptide.

The compositions provided herein can be formulated as neutral or salt forms. Pharmaceutically acceptable salts include those formed with anions such as those derived from hydrochloric, phosphoric, acetic, oxalic, tartaric acids, etc., and those formed with cations such as those derived from sodium, potassium, ammonium, calcium, ferric hydroxides, isopropylamine, triethylamine, 2-ethylamino ethanol, histidine, procaine, etc.

The amount of a prophylactic or therapeutic agent (e.g., a peptide provided herein), or a composition provided herein that will be effective in the prevention and/or treatment of a disease or condition can be determined by standard clinical techniques. In addition, in vitro assays may optionally be employed to help identify optimal dosage ranges. The precise dose to be employed in the formulation will also depend on the route of administration, and the seriousness of a disease or condition, and should be decided according to the judgment of the practitioner and each patient's circumstances.

Effective doses may be extrapolated from dose-response curves derived from in vitro or animal model test systems.

In certain embodiments, the route of administration for a dose of a peptide provided herein to a patient is intranasal, intramuscular, intravenous, or a combination thereof, but other routes described herein are also acceptable. Each dose may or may not be administered by an identical route of administration. In some embodiments, a peptide provided herein may be administered via multiple routes of administration simultaneously or subsequently to other doses of the same or a different peptide provided herein.

In certain embodiments, the peptide provided herein are administered prophylactically or therapeutically to a subject. The peptide provided herein can be prophylactically or therapeutically administered to a subject so as to prevent, lessen or ameliorate a disease or symptom thereof.

### 5.7 Kits

The peptides provided herein can also be provided as an article of manufacture using packaging materials well known to those of skill in the art. See U.S. Pat. Nos. 5,323,907; 5,052,558; and 5,033,252. Examples of pharmaceutical packaging materials include, but are not limited to, blister packs, bottles, tubes, inhalers, pumps, bags, vials, containers, syringes, and any packaging material suitable for a selected formulation and intended mode of administration and treatment.

In certain embodiments, provided herein also are kits which, when used by the medical practitioner, can simplify the administration of appropriate amounts of active ingredients to a subject. In certain embodiments, the kit provided herein includes a container and a dosage form of a peptide provided herein or a pharmaceutical composition comprising same.

In certain embodiments, the kit includes a container comprising a dosage form of a peptide provided herein or a pharmaceutical composition comprising same in a container comprising one or more other therapeutic agent(s) described herein.

Kits provided herein can further include devices that are used to administer the active ingredients. Examples of such devices include, but are not limited to, syringes, needle-less injectors drip bags, patches, and inhalers. The kits provided herein can also include condoms for administration of an active ingredient.

Kits provided herein can further include pharmaceutically acceptable vehicles that can be used to administer one or more active ingredients. For example, if an active ingredient is provided in a solid form that must be reconstituted for parenteral administration, the kit can comprise a sealed container of a suitable vehicle in which the active ingredient can be dissolved to form a particulate-free sterile solution that is suitable for parenteral administration. Examples of pharmaceutically acceptable vehicles include, but are not limited to: aqueous vehicles, including, but not limited to, Water for Injection USP, Sodium Chloride Injection, Ringer's Injection, Dextrose Injection, Dextrose and Sodium Chloride Injection, and Lactated Ringer's Injection; water-miscible vehicles, including, but not limited to, ethyl alcohol, polyethylene glycol, and polypropylene glycol; and non-aqueous vehicles, including, but not limited to, corn oil, cottonseed oil, peanut oil, sesame oil, ethyl oleate, isopropyl myristate, and benzyl benzoate.

For the sake of conciseness, certain abbreviations are used herein. One example is the single letter abbreviation to represent amino acid residues. The amino acids and their corresponding three letter and single letter abbreviations are as follows:

| | | |
|---|---|---|
| alanine | Ala | (A) |
| arginine | Arg | (R) |
| asparagine | Asn | (N) |
| aspartic acid | Asp | (D) |
| cysteine | Cys | (C) |
| glutamic acid | Glu | (E) |
| glutamine | Gln | (Q) |
| glycine | Gly | (G) |
| histidine | His | (H) |
| isoleucine | Ile | (I) |
| leucine | Leu | (L) |
| lysine | Lys | (K) |
| methionine | Met | (M) |
| phenylalanine | Phe | (F) |
| proline | Pro | (P) |
| serine | Ser | (S) |
| threonine | Thr | (T) |
| tryptophan | Trp | (W) |
| tyrosine | Tyr | (Y) |
| valine | Val | (V) |

The invention is generally disclosed herein using affirmative language to describe the numerous embodiments. The invention also specifically includes embodiments in which particular subject matter is excluded, in full or in part, such as substances or materials, method steps and conditions, protocols, procedures, assays or analysis. Thus, even though the invention is generally not expressed herein in terms of what the invention does not include, aspects that are not expressly included in the invention are nevertheless disclosed herein.

### 6. EXAMPLES

The following is a description of various methods and materials used in the studies, and are put forth so as to provide those of ordinary skill in the art with a complete disclosure and description of how to make and use the present invention, and are not intended to limit the scope of what the inventors regard as their invention nor are they intended to represent that the experiments below were performed and are all of the experiments that may be performed. It is to be understood that exemplary descriptions written in the present tense were not necessarily performed, but rather that the descriptions can be performed to generate the data and the like associated with the teachings of the present invention. Efforts have been made to ensure accuracy with respect to numbers used (e.g., amounts, percentages, etc.), but some experimental errors and deviations should be accounted for.

### 6.1 Example 1—Generation of Variants of P8 Peptides

P8 peptide and its variants were synthesized by solid-phase peptide synthesis. Peptides were prepared by using p-benzyloxy-benzylalcohol resin (Wang resin). All amino acids were coupled as 9-fluorenylmethoxycarbonyl (Fmoc)-derivatives (Nova Biochem). The tert-butyl group was applied as a protecting group for the side chain. For 8M2 and 8M2D, the acetylation modification was carried out by adding a solution of 20% acetic anhydride to the resin. For 8M1, 8M2, 8M1D, and 8M2D, amidation was carried out by adding Hexafluorophosphate Azabenzotriazole Tetramethyl Uronium (HATU) and diisopropylethylamine (DIPEA), then proceeded by passing through ammonia gas. After the completion of the synthesis, the peptides were cleaved from the resin. The crude products were purified by high performance liquid chromatography on a reverse-phase column (RP-HPLC) to an extent greater than about 98% prior to use.

TABLE 1
Peptide Sequences of P8 and Its Variant Peptides

| | | Molecular | | |
|---|---|---|---|---|
| Peptide | | Weight | | SEQ |
| name | Sequence | (g/mol) | Notes | ID NO. |
| P8 | DEEEDEEL | 1,007 | Parent | 1 |
| | | | peptide | |
| 8M1 | DEEEDEEL—NH2 | 1,006 | Modification | 2 |
| | | | of P8 | |
| 8M2 | Ac- | | Modification | 3 |
| | DEEEDEEL—NH2 | | of P8 | |
| 8M1D | dEEEDEEL—NH2 | 1,006 | Modification | 4 |
| | | | of P8 | |
| 8M2D | Ac- | | Modification | 5 |
| | dEEEDEEL—NH2 | | of P8 | |

Note:
—NH2 means C-terminal Amidation(—CONH₂);
Ac means N-terminal Acetylation;
d means D-amino acid

### 6.2 Example 2—Metabolic Stability of Peptides in Fresh Human Plasma

P8, 8M1, and 8M1D were generated according to Example 1 with purities ranging from 98.0% to 99.0%. A primary 1.00 mM stock solution was prepared in either acetonitrile or acetonitrile:water (1:1, v/v) for each peptide. A 0.200 mM working stock was made from the primary stock in acetonitrile:water (1:1, v/v), which was used for the reactions. The primary and working stock solutions were stored at −20° C. when not in use, and they were kept at room temperature for as short a time as possible when in use. Fresh human plasma were maintained by adding sodium heparin as an anticoagulant.

The incubation procedures started when 5.00 μL of 0.2 mM (working stock solutions) of different peptides were added to 0.995 mL of fresh human plasma in a 1.7-mL snap tube. After 3, 8 and 24 hours of incubation, duplicate 50.0 μL aliquots of plasma were removed and placed into extraction tubes containing 150 μL of methanol, which served to quench any reactions, and thus incubations were ended. For each time point, the aliquotes were immediately extracted by vortex mixing and centrifugation. Resulting supernates were stored in High-Performance Liquid Chromatography (HPLC) vials and analyzed by LC/MS/MS after all samples were extracted. Valacyclovir was used as a positive control.

LC/MS/MS analysis of the incubation solutions was conducted by initial separation of the test article peaks using chromatography prior to detection by the mass spectrometer. The LC/MS system was comprised of a HPLC coupled with a TQS-Micro or Quattro Premier (Waters, Milford, Mass.). The mobile phase was nebulized using heated nitrogen in a Z spray source/interface set to electrospray in positive ionization mode for the peptides. The ionized peptides were detected using Tandem Quadropole Mass Spectrometry (MS/MS). The data was acquired using MassLynx (Waters, Milford, Mass.).

HPLC for the peptides and Valacyclovir control were performed with no guard columns, with a flow rate at 0.3 mL/min, with column temperature at 40° C., and autosampler temperature at 10° C. The column for peptides was MacMod ACE 2 Excel C18PFP, 100×2.1-mm, 2.0 μm, and the column for the control was Waters Atlantis T3, 150×2.1-mm, 5.0 μm. The injection loop and volumn for peptides were 50.0 μL and 20.0 μL respectively, and the ones for controls were 20.0 μL and 2.00 μL. For the Mass Spectrometer for the peptides and the Valacyclovir control, the source temperature was set to be 150° C., desolvation temperature was set to be 350° C., and polarity was set to be positive mode of electrospray ionization. Other parameters in HPLC and Mass Spectrometer are shown in Table 2.1, Table 2.2 and Table 3 below.

TABLE 2.1
HPLC Linear Gradient Program for Peptides

| Time(min) | Solvent A | Solvent B | curve |
|---|---|---|---|
| 0.00-0.50 | 80% | 20% | 6 |
| 0.50-2.00 | Decrease to 5% | Increase to 95% | 6 |
| 2.00-3.00 | 5% | 95% | 6 |
| 3.00-3.10 | Increase to 80% | Decrease to 20% | 6 |
| 3.10-6.00 | 80% | 20% | 6 |

TABLE 2.2
HPLC Linear Gradient Program for Valacyclovir

| Time(min) | Solvent A | Solvent B | curve |
|---|---|---|---|
| 0.00 | 90% | 10% | 6 |
| 0.00-1.50 | Decrease to 40% | Increase to 60% | 6 |
| 1.50-1.60 | Decrease to 5% | Increase to 95% | 6 |
| 1.60-2.00 | 5% | 95% | 6 |
| 2.00-2.10 | Increase to 90% | Decrease to 10% | 6 |
| 2.10-4.00 | 90% | 10% | 6 |

Note:
Solent A is 0.1% Formic Acid in Water; Solvent B is 0.1% Formic Acid in Methanol

TABLE 3
Mass Spectrometer Conditions for Peptides and Valacyclovir

| | | | Collision |
|---|---|---|---|
| Analyst | Mass Transition | Cone Energy (V) | Energy (eV) |
| P8 | 1,007.5 > 876.4 | 10 | 25 |
| 8M1 | 1,006.6 > 876.4 | 10 | 30 |
| 8M1D | 1,006.5 > 876.4 | 25 | 35 |
| Valacyclovir | 325.1 > 152.0 | 5 | 15 |

The results of LC/MS/MS analysis are summarized in Table 4 below, which reveals that different peptides were metabolized in fresh human plasma at different rates, with 8M1D the slowest, and 8M1 the fastest. Specifically, the peptides P8 and 8M1D were relatively stable in fresh human plasma, with 8M1D outperforming P8.

TABLE 4
Results of Metabolic Stability Assay of P8, 8M1, and 8M1D

| | Time | Peak | Mean | |
|---|---|---|---|---|
| Peptide | (h) | Height | Height | % Remaining |
| 8M1 | 0 | 12,282 | 12,093 | 100 |
| | | 11,904 | | |
| | 3 | 12,038 | 10,539 | 87.1 |
| | | 9,040 | | |
| | 8 | 8,129 | 7,874 | 65.1 |
| | | 7,618 | | |
| | 24 | 5,047 | 5,693 | 47.1 |
| | | 6,339 | | |
| 8M1D | 0 | 15,325 | 16,043 | 100 |
| | | 16,760 | | |
| | 3 | 17,259 | 18,474 | 115.2 |
| | | 19,688 | | |
| | 8 | 17,405 | 16,111 | 100.4 |
| | | 14,817 | | |
| | 24 | 15,683 | 14,367 | 89.6 |
| | | 13,051 | | |
| P8 | 0 | 10,793 | 10,043 | 100 |
| | | 9,293 | | |
| | 3 | 10,026 | 11,540 | 115 |
| | | 13,054 | | |
| | 8 | 7,629 | 10,011 | 99.7 |
| | | 12,392 | | |
| | 24 | 7,948 | 7,241 | 72 |
| | | 6,533 | | |

### 6.3 Example 3—Pharmacokinetics Study in Rats

An in-vivo study was carried out in rats to determine the pharmacokinetics (PK) of peptides P8, 8M1D and 8M2D in plasma and CSF. Each peptide was assessed through two dosage routes, intravenous (IV) and subcutaneous (SC). Three rats were assigned to each dose group with each peptide. Total of twelve rats per peptide were used. The study design is shown in Table 5.

TABLE 5
Design for Pharmacokinetics Study in Rats for Each Peptide

| Dose | Dosing | | Dose | Blood Sampling | CSF Sampling |
|---|---|---|---|---|---|
| Group | Route | N= | (mg/kg) | Time points | Time points |
| 1 | IV | 3 | 10 mg/kg | Pre-dose, 10, 30 min, 1, | Predose, 1, 4, and 12 hr |
| | | | | 2, 4, 6, 8, 12, and 24 hrs | |
| 2 | IV | 3 | 10 mg/kg | Pre-dose, 10, 30 min, 1, | 30 mins, 2, 8, and 24 hr |
| | | | | 2, 4, 6, 8, 12, and 24 hrs | |
| 3 | SC | 3 | 10 mg/kg | Pre-dose, 10, 30 min, 1, | Predose, 1, 4, and 12 hr |
| | | | | 2, 4, 6, 8, 12, and 24 hrs | |
| 4 | SC | 3 | 10 mg/kg | Pre-dose, 10, 30 min, 1, | 30 mins, 2, 8, and 24 hr |
| | | | | 2, 4, 6, 8, 12, and 24 hrs | |

Blood was collected through jugular vein cannulas, and the samples were collected into tubes with K₂EDTA. After centrifuging at a temperature of 2 to 8° C. at 4,000×g for 5 minutes, the resulting plasma was split into equal aliquots, frozen, and stored at −60 to −80° C. One aliquot was for PK analysis and the other was for Aβ analysis (see Example 4).

CSF collection was performed by first anesthetizing animals at specified time points (see Table 5) with a mixture of 80 mg/kg ketamine and 8 mg/kg xylazine. The depth of anesthesia was monitored by toe pinches. CSF was collected via the cisterna magna (˜0.05 mL), and it was split into equal aliquots, frozen, and stored at −60 to −80° C.

Both 8M1D and 8M2D had better exposures than P8 in the plasma at Cmax and most subsequent time points (see Tables 6, 7, and 8 below). Exposure was slightly higher with 8M1D than with 8M2D. 8M1D and 8M2D were also detectable in plasma longer than P8. Specifically, 8M1D and 8M2D were still detectable at 4 h and 6 h after dosing, while P8 was not detectable after 2 h.

TABLE 6
Results of the PK Study of P8 in Rats

| | Rat I.D. | | | | | |
|---|---|---|---|---|---|---|
| | Group 1-P8; 10 mg/kg IV | | | Group 3-P8; 10 mg/kg SC | | |
| Time (hr) | 821 | 822 | 823 | 827 | 828 | 829 |
| 0 | BQL | BQL | BQL | BQL | BQL | BQL |
| 0.167 | 9,770 | 12,800 ^{a} | 11,800 ^{a} | 5,610 | 14,400 ^{a} | 11,900 ^{a} |
| 0.5 | 2,340 | 2,080 | 2,710 | 3,030 | 7,010 | 7,250 |
| 1 | 257 | 219 | 189 | 670 | 1,300 | 1,970 |
| 2 | 26.3 | 22.7 | 18.9 | 105 | 191 | 245 |
| 4 | 3.23 ^{b} | 3.18 ^{b} | 2.89 ^{b} | 5.44 ^{b} | 9.41 ^{b} | 20.7 |
| 6 | 1.07 ^{b} | — | 0.794 ^{b} | 1.18 ^{b} | 4.35 ^{b} | 9.33 ^{b} |
| 8 | BQL | — | BQL | BQL | 1.32 ^{b} | 1.51 ^{b} |
| 12 | BQL | — | BQL | BQL | BQL | 0.900 ^{b} |
| 24 | BQL | — | BQL | BQL | — | BQL |

TABLE 7
Results of the PK Study of 8M1D in Rats

| | Rat I.D. | | | | | |
|---|---|---|---|---|---|---|
| | Group 1-8M1D; 10 mg/kg IV | | | Group 3-8M1D; 10 mg/kg SC | | |
| Time (hr) | 833 | 834 | 835 | 839 | 840 | 841 |
| 0 | BQL | BQL | BQL | BQL | BQL | BQL |
| 0.167 | 25,600 | 23,000 | 20,200 | 20,500 ^{a} | 19,100 | 25,900 |
| 0.500 | 7,140 | 8,630 | 6,650 | 12,300 ^{a} | 10,900 | 11,800 |
| 1 | 1,680 | 2,160 | 1,580 | 5,440 | 4,310 | 3,410 |
| 2 | 215 | 360 | 290 | 1,330 | 1,570 | 642 |
| 4 | 25.6 | 16.0 | 17.0 | 49.8 | 75.3 | 43.9 |
| 6 | 2.93 ^{b} | 0.805 ^{b} | 1.48 ^{b} | 11.4 | 8.83 ^{b} | 4.66 ^{b} |
| 8 | BQL | BQL | BQL | 1.29 ^{b} | BQL | BQL |
| 12 | BQL | BQL | BQL | BQL | BQL | BQL |
| 24 | BQL | BQL | BQL | BQL | BQL | BQL |

TABLE 8
Results of the PK Study of 8M2D in Rats

| | Rat I.D. | | | | | |
|---|---|---|---|---|---|---|
| | Group 1-8M2D; 10 mg/kg IV | | | Group 3-8M2D; 10 mg/kg SC | | |
| Time (hr) | 884 | 885 | 886 | 890 | 891 | 892 |
| 0 | BQL | BQL | BQL | BQL | BQL | BQL |
| 0.167 | 20,900 | 21,000 | 23,400 | 16,200 | 13,500 | 13,800 ^{a} |
| 0.500 | 5,620 | 5,680 | 5,940 | 9,030 | 9,010 | 7,190 |
| 1 | 1,450 | 1,040 | 1,190 | 3,250 | 4,160 | 3,110 |
| 2 | 417 | 114 | 136 | 609 | 987 | 1,040 |
| 4 | 10.2 | 7.54 ^{b} | 8.29 ^{b} | 59.2 | 82.8 | 125 |
| 6 | 2.09 ^{b} | 1.81 ^{b} | BQL | 3.77 ^{b} | 9.84 ^{b} | 21.6 |
| 8 | BQL | BQL | BQL | 1.61 ^{b} | 4.37 ^{b} | 5.19 ^{b} |
| 12 | BQL | BQL | BQL | BQL | BQL | BQL |
| 24 | BQL | BQL | BQL | — | BQL | BQL |

As shown in Tables 9, 10, and 11, both 8M1D and 8M2D had better exposures than P8 in the CSF at Cmax and most subsequent time points. Exposure in the CSF was higher with 8M2D than with 8M1D. 8M1D and 8M2D were also detectable in CSF for much longer time than P8. Specifically, 8M1D was still detectable at 8 h, and 8M2D was still detectable at 12 h after dosing, while P8 was not detectable after 2 h.

TABLE 9
Results of the PK Study of P8 in Rats

| Time (hr) | Mean IV | Mean SC |
|---|---|---|
| 0 | 0 | 0 |
| 0.5 | 42.4 | 79.4 |
| 1 | 14.2 | 19.3 |
| 2 | 4.89 | 23.3 |
| 4 | 0 | 0 |
| 8 | 0 | 0 |
| 12 | 0 | 0 |
| 24 | 0 | 0 |

TABLE 10
Results of the PK Study of 8M1D in Rats

| Time (hr) | Mean IV | Mean SC |
|---|---|---|
| 0 | 0 | 0 |
| 0.5 | 67.8 | 50.9 |
| 1 | 80.8 | 66.1 |
| 2 | 65 | 62.9 |
| 4 | 23.4 | 18.9 |
| 8 | 0.913 | 3.85 |
| 12 | 0 | 0 |
| 24 | 0.261 | 0 |

TABLE 11
Results of the PK Study of 8M2D in Rats

| Time (hr) | Mean IV | Mean SC |
|---|---|---|
| 0 | 0 | 0 |
| 0.5 | 104 | 59 |
| 1 | 55.6 | 53.4 |
| 2 | 68 | 172 |
| 4 | 11.5 | 13.3 |
| 8 | 1.75 | 15.3 |
| 12 | 0.378 | 0.113 |
| 24 | 1.2 | 0 |

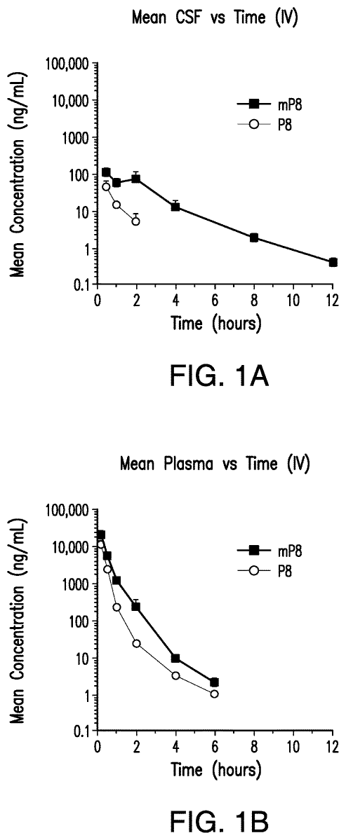

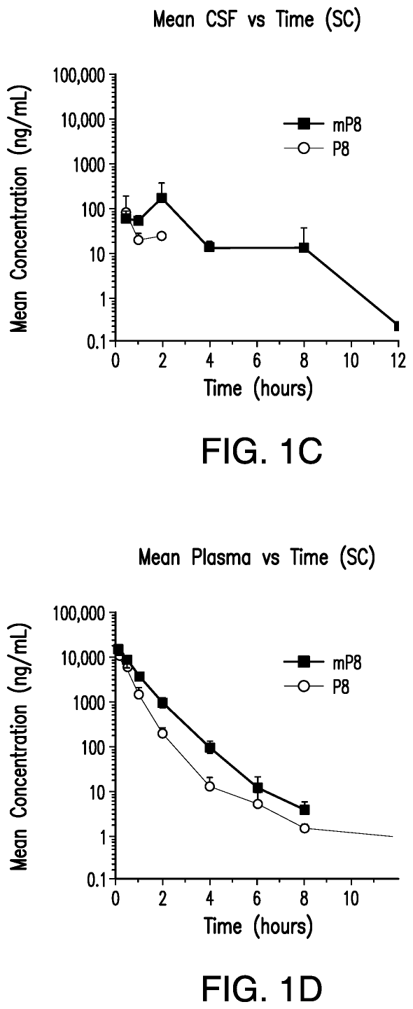

Pharmacokinetics (PK) of peptides P8 and 8M2D were particularly compared in plasma and CSF. Similarly, each test article was assessed through two dosage routes, IV and SC. N=3 per dose group per test article. Results are shown in FIGS. 1A-1D. As shown, in plasma 8M2D had better exposures than P8 at Cmax and most subsequent time points. 8M2D was also detectable in plasma longer than P8 (at 4 h and 6 h after dosing compared to P8, which was no longer detectable 2 h post-dosing). Similarly, in CSF 8M2D had higher exposure than P8 at Cmax and most subsequent time points. 8M2D was also detectable in CSF for much longer than P8 (up to 12 h post-dosing compared to P8, which was no longer detectable 2 h post-dosing).

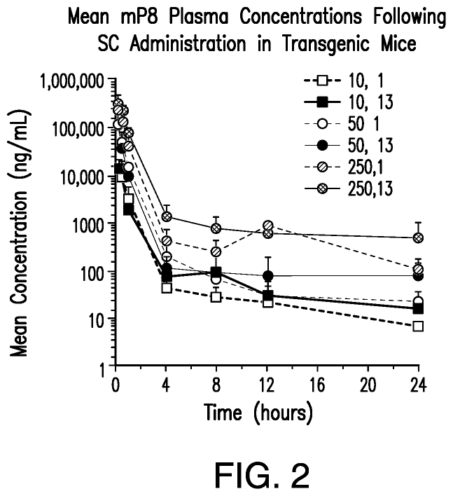

Pharmacokinetics (PK) of 8M2D was further analyzed with various doses at days 1 and 13 in transgenic mice (APPSWE (B6; SJL 2576 Kha; Taconic)). See Hsiao et al., Correlative memory deficits, Aβ elevation, and amyloid plaques in transgenic mice. Science 274:99-102 (1996); and Haugabook et al., Reduction of Abeta accumulation in the Tg2576 animal model of Alzheimer's disease after oral administration of the phosphatidyl-inositol kinase inhibitor wortmannin. Faseb J 15:16-18 (2001). The results are shown in Table 12 and FIG. 2.

TABLE 12
PK Analysis of 8M2D in Transgenic Mice

| | Dose | Tₘₐₓ | Cₘₐₓ | AUCₗₐₛₜ | Vz/F | Cl/F | t_{1/2} |
|---|---|---|---|---|---|---|---|
| Day | (mg/kg) | (h) | (ng/mL) | (h*ng/mL) | (L/kg) | (mL/min/kg) | (h) |
| 1 | 10 | 0.17 | 14400 | 12800 | 8.84 | 13.0 | 7.9 |
| | 50 | 0.17 | 111000 | 70500 | 7.59 | 11.8 | 7.4 |
| | 250 | 0.17 | 207000 | 176000 | 5.84 | 23.6 | 2.9 |
| | N | 3 | 3 | 3 | 3 | 3 | 3 |
| | Mean | 0.167 | 111000 | 86400 | 7.42 | 16.1 | 6.06 |
| | SD | 0.00 | 96400 | 82800 | 1.50 | 6.52 | 2.78 |
| | CV % | 0.00 | 87.0 | 95.8 | 20.3 | 40.5 | 45.9 |
| 13 | 10 | 0.17 | 12400 | 11600 | 10.2 | 14.1 | 8.4 |
| | 50 | 0.17 | 73000 | 49700 | 148 | 12.9 | 130 |
| | 250 | 0.17 | 278000 | 290000 | 33.4 | 13.5 | 29 |
| | N | 3 | 3 | 3 | 3 | 3 | 3 |
| | Mean | 0.167 | 121000 | 117000 | 63.8 | 13.5 | 56.5 |
| | SD | 0.00 | 139000 | 151000 | 73.6 | 0.610 | 66.6 |
| | CV % | 0.00 | 115 | 129 | 115 | 4.52 | 118 |

### 6.4 Example 4—Efficacy of P8, 8M1D and 8M2D in Reducing Aβ40 in Rat Plasma

Rats (n=3 per peptide) were administered with P8, 8M1D, or 8M2D (10 mg/kg) subcutaneously, and plasma were collected at 0, 6, 12, and 24 h after dosing and Aβ40 analysis was carried out by ELISA (Invitrogen).

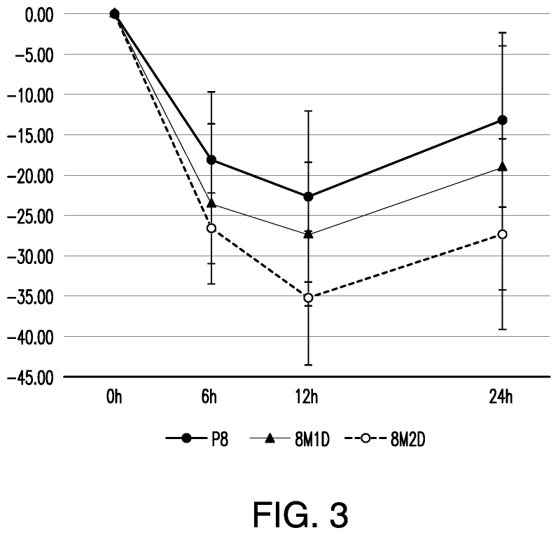

Results (see FIG. 3) revealed that 8M2D was more potent than P8 and 8M1D in that it reduced the amount of Aβ40 to a greater extent. Specifically, at 12 hr post dosing, 8M2D reduced Aβ40 by about 35%; while P8 reduced Aβ40 by about 22% and 8M1D reduced Aβ40 by about 27%. At 24 h post dosing, 8M2D still showed a long-lasting effect on reduction in Aβ40 at about 27%, which was more than doubled when compared to P8, which caused about 13% reduction in Aβ40. 8M2D was also superior to 8M1D, which caused about 19% reduction in Aβ40 at 24 hours.

TABLE 13
Percent Decrease in Aβ 40 in Rat Plasma Treated with P8,
8M1D and 8M2D (Results show mean values for 3 rats per peptide)

| Sample ID | 0 h | 6 h | 12 h | 24 h |
|---|---|---|---|---|
| P8 | 0.00 | −18.13 | −22.70 | −13.22 |
| 8M1D | 0.00 | −23.61 | −27.34 | −19.13 |
| 8M2D | 0.00 | −26.62 | −35.27 | −27.36 |

Similar studies are performed to analyze the efficiency of the peptides at reducing Aβ42.

### 6.5 Example 5—Delivery to Brain and Efficacy of P8 and 8M2D in Reducing Aβ in Transgenic Mice

Critical to the development of any therapeutic for diseases like AD is the requirement that it can be delivered to the brain. Delivery to brain and efficacy of P8 and 8M2D in reducing Aß was analyzed in the transgenic mice described in Section 6.3 above.

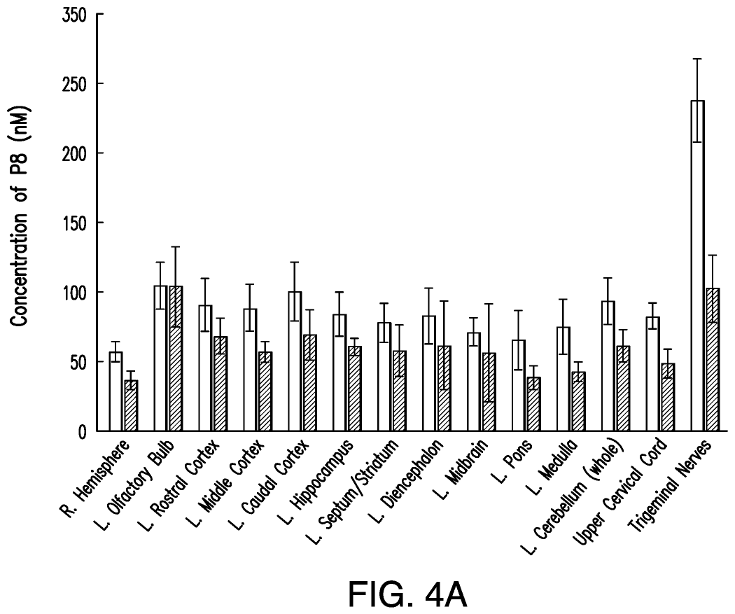

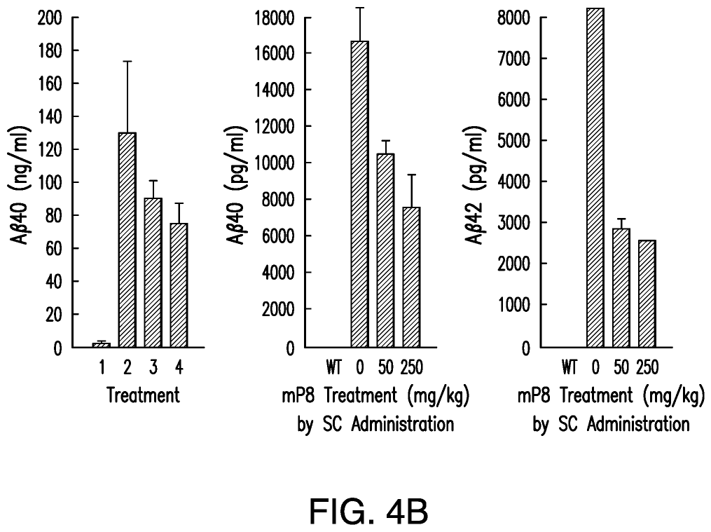

As shown in FIGS. 4A-4B and Table 14 below, SC administration delivers P8 and 8M2D to the APPTg mouse brain, and that it does so in sufficient amounts to reduce Aß in the brain. Specifically, Aß analysis in CSF of APP Transgenic mice following P8 (FIG. 4A) and 8M2D (FIG. 4B) administration shows target engagement, brain penetration and efficacy using SC dosing (analysis in CSF and brain). The maximum efficacious dose of 8M2D in Tg mice over-expressing Aß is at just over 50 mg/kg. This gives a 65% reduction in Aß 42. Increasing the dose 5× gives only a marginal increase in efficacy. Experiments are performed to establish the minimum efficacious dose in Tg mice.

TABLE 14
Brain Concentrations

| Animal | Brain Concentration (mg/g) by Dose (mg/kg) | | |
|---|---|---|---|
| ID | 10 | 50 | 250 |
| 11 | BQL | | |
| 12 | BQL | | |
| 13 | BQL | | |
| 14 | BQL | | |
| 15 | | 9.38 | |
| 13 | | 8.73 | |
| 17 | | BQL | |
| 18 | | BQL | |
| 19 | | BQL | |
| 20 | | | 5.10 |
| 21 | | | 14.6 |
| 22 | | | 16.9 |
| 23 | | | 19.9 |
| 24 | | | 30.3 |
| N | 0 | 2 | 5 |
| Mean | | 9.06 | 15.4 |
| SD | | 0.460 | 6.19 |
| CV % | | 5.08 | 40.3 |

### 6.6 Example 6—Reduction of Aß in Retina

The accumulation of Aβ in the retina was studied in this example. Retinal cells were differentiated from induced pluripotent stem (iPSC) cells derived from Alzheimer's disease patients. The results indicate that these retinal cells show increased production of AB, which can be reduced in the presence of P8 or 8M2D.

Similar experiments are performed to test the functions of P8 and 8M2D in reducing Aß in retinal cells from patients having age-related macular degeneration.

What is claimed:

1. A peptide comprising
(i) a sequence of Ac-DEEEDEEL-NH₂ (SEQ ID NO:3), wherein the N-terminal amino acid Asp is acetylated and the C-terminal amino acid Leu is amidated;
(ii) a sequence of dEEEDEEL-NH₂ (SEQ ID NO:4), wherein the N-terminal amino acid Asp is a D-amino acid and the C-terminal amino acid Leu is amidated; or
(iii) a sequence of Ac-dEEEDEEL-NH₂ (SEQ ID NO:5), wherein the N-terminal amino acid Asp is a D-amino acid and is acetylated and the C-terminal amino acid Leu is amidated.

2. The peptide of claim 1, wherein the peptide comprises a sequence of Ac-DEEEDEEL-NH₂ (SEQ ID NO:3).

3. The peptide of claim 1, wherein the peptide comprises a sequence of dEEEDEEL-NH₂ (SEQ ID NO:4).

4. The peptide of claim 1, wherein the peptide comprises a sequence of Ac-dEEEDEEL-NH₂ (SEQ ID NO:5).

5. A peptide comprising a first domain comprising a sequence of SEQ ID NO: 3, SEQ ID NO:4 or SEQ ID NO:5, and a second domain.

6. The peptide of claim 5, wherein the second domain comprises an Fc domain.

7. The peptide of claim 5, wherein the second domain comprises a purification peptide.

8. A pharmaceutical composition comprising the peptide of any one of claims 1 to 7 and a pharmaceutically acceptable excipient.

9. A method of attenuating the binding of beta-Amyloid precursor protein (β-APP) with presenilin-1 (PS-1) and/or presenilin-2 (PS-2) in a cell comprising contacting a cell with the peptide of any one of claims 1 to 7 or the pharmaceutical composition of claim 8.

10. A method of attenuating the production of amyloid β, attenuating an amyloid β activity, attenuating the production of tau protein, or attenuating an tau protein activity in a cell comprising contacting the cell with the peptide of any one of claims 1 to 7 or the pharmaceutical composition of claim 8, wherein optionally the amyloid β activity is an amyloid β induced signaling, and wherein optionally the tau protein activity is a tau protein-induced signaling.

11. A method of attenuating the production of amyloid β, attenuating an amyloid β activity, attenuating the production of tau protein, or attenuating an tau protein activity in a subject comprising administering a therapeutically effective amount of the peptide of any one of claims 1 to 7 or the pharmaceutical composition of claim 8 to the subject, wherein optionally the amyloid β activity is an amyloid β induced signaling, and wherein optionally the tau protein activity is a tau protein-induced signaling.

12. The method of claim 10 or 11, wherein the amyloid β is amyloid β 40.

13. The method of claim 10 or 11, wherein the amyloid β is amyloid β 42.

14. A method of treating a disease or disorder in a subject, comprising administering to the subject a therapeutically effective amount of the peptide of any one of claims 1 to 7 or the pharmaceutical composition of claim 8.

15. The method of claim 14, wherein the disease or disorder is an amyloid (or amyloid β) related disease or disorder, a disease or disorder associated with amyloid fibril formation, aggregation or deposition, a neurological disease, or a neurodegenerative disease.

16. The method of claim 14, wherein the disease or disorder is selected from a group consisting of Alzheimer's disease, Parkinson's disease, traumatic brain injury, amyotrophic lateral sclerosis, multiple sclerosis, and dementia.

17. The method of claim 14, wherein the disease or disorder is dementia or a dementia related disease or disorder selected from a group consisting of frontotemporal dementia, fronto-temporal degeneration associated with Pick's disease, vascular dementia, corticobasal degeneration, ischemic vascular dementia (IVD), Lewy body dementia, and Alzheimer's dementia.

18. The method of claim 14, wherein the disease or disorder is an ocular disorder or Down syndrome.

19. The method of claim 18, wherein the ocular disorder is related to Alzheimer's disease.

20. The method of claim 18, wherein the ocular disorder is macular degeneration, and wherein optionally, the macular degeneration is age-related macular degeneration (AMD).

21. The method of claim 14, wherein the disease or disorder is selected from a group consisting of transmissible spongiform encephalopathies, cerebral amyloid angiopathy, hereditary cerebral hemorrhage with amyloidosis, mild cognitive impairment, sporadic inclusion body myositis and age-related macular degeneration.

22. A method for improving memory in a subject, comprising administering to a subject a therapeutically effective amount of the peptide of any one of claims 1 to 7 or the pharmaceutical composition of claim 8.

23. The method of any one of claims 11 to 22, wherein the subject is a human subject.


SEQUENCE LISTING (5 sequences)

SEQ ID NO: 1 - protein, 8 aa, Artificial Sequence
  Parent peptide of P8
DEEEDEEL

SEQ ID NO: 2 - protein, 8 aa, Artificial Sequence
  Modification of peptide P8 - 8M1
  MOD_RES (8)..(8): AMIDATION
DEEEDEEL

SEQ ID NO: 3 - protein, 8 aa, Artificial Sequence
  Modification of peptide P8 - 8M2
  MOD_RES (1)..(1): ACETYLATION
  MOD_RES (8)..(8): AMIDATION
DEEEDEEL

SEQ ID NO: 4 - protein, 8 aa, Artificial Sequence
  Modification of peptide P8 - 8M1D
  MOD_RES (1)..(1): Xaa = D-Asp
  MOD_RES (8)..(8): AMIDATION
XEEEDEEL

SEQ ID NO: 5 - protein, 8 aa, Artificial Sequence
  Modification of peptide P8 - 8M2D
  MOD_RES (1)..(1): ACETYLATION
  MOD_RES (1)..(1): Xaa = D-Asp
  MOD_RES (8)..(8): AMIDATION
XEEEDEEL